(19) United States
(12) Patent Application Publication
(10) Pub. No.: US 2026/0061117 A1
(43) Pub. Date: Mar. 5, 2026

(54) FLUID ASSIST ARRANGEMENT FOR DETERMINING A FLUID CHARACTERISTIC OF A FLUID

(71) Applicant: OSAA INNOVATION APS, Hillerød (DK)

(72) Inventor: Ahmed Abdullah HESSAM, Hillerød (DK)

(21) Appl. No.: 19/312,942

(22) Filed: Aug. 28, 2025

(30) Foreign Application Priority Data
Sep. 2, 2024 (DK) PA 2024 30513
Jul. 23, 2025 (DK) PA 2025 30482

(51) Int. Cl. A61M 5/14; A61M 5/168

(52) U.S. Cl. CPC A61M 5/1411; A61M 5/16877; A61M 5/16886; A61M 2205/3306

(57) ABSTRACT

The fluid assist arrangement includes a monitoring device. The monitoring device includes a body having a front side and a back side. The front side of the body includes a body recess. The monitoring device also includes one or more light emitting elements as well as one or more light receiving elements. Both the one or more light emitting elements and the one or more light receiving elements are arranged in the body recess. The one or more light emitting elements are configured to emit at least one beam having a beam axis.

### CROSS-REFERENCE TO RELATED APPLICATIONS

This application claims the benefit of Danish Application Nos. PA 2024 30513, filed 2024-09-02, and PA 2025 30482, filed 2025-07-23, the entire disclosures of which are hereby incorporated herein by reference.

### FIELD OF THE INVENTION

The present invention relates to a fluid assist arrangement for determining a fluid characteristic of a fluid. More specifically, the present invention relates to a fluid assist arrangement for determining a fluid characteristic of a fluid in an IV system.

### BACKGROUND

Intravenous (IV) fluid systems are used to deliver a predetermined volume of fluids directly into the vein of individuals within a predetermined timeslot and are one of the most commonly used medical devices used by healthcare professionals. IV fluid systems are for example used to deliver nutrients, vitamins, blood, and medication. A typical IV fluid system includes an IV bag, a drip chamber connected to the IV bag through a tube and a cannula connected to the drip chamber through a second tube.

IV fluid systems often operate based on gravity, where a user manually adjust the flow rate of the fluid by counting the number of drops of the fluid within the drip chamber and based on the number of drops adjusts a roller clamp attached to the tube of the IV fluid system. Thus, the efficiency of the IV fluid system to deliver a predetermined volume of fluid within a predetermined timeslot depends on the accuracy of the user to both determine and control the fluid flow rate during the delivery of the fluid. In some cases, it may be necessary to adjust the fluid flow rate multiple times during delivery of the fluid, to get the desired response from the individual.

WO2019142125A1 discloses a device comprising a housing defining a channel there through, a torsion-spring based clamping mechanism, a drop sensor connected with circuitry, a manual infusion fluid regulation mechanism and a power source. An upper portion of the channel accommodates at least a part of a drip chamber and a lower portion of the channel accommodates at least a part of infusion tubing. The clamping mechanism comprises of one or more torsion springs and is configured for engaging and holding drip chamber and can be adjusted according to the drip chamber diameter. The drop sensor and the circuits coupled with it are configured for detecting real time drop rate of the infusion fluid. The regulation mechanism comprises of a drivable pinching element for sliding into the channel and compressing the infusion tubing thereby regulating the flow of the infusion fluid through the infusion tubing.

### DISCLOSURE OF THE INVENTION

An object of the present invention is to provide a fluid assist arrangement for assisting dispensing of a fluid in a fluid system in which a fluid characteristic of a fluid effectively can be determined to ensure a safe dispensing of the fluid.

In an embodiment, it is an object of the present invention to provide a fluid assist arrangement with high reliability and control of a fluid dispensed through a fluid system.

In an embodiment, it is an object of the present invention to provide a fluid assist arrangement which effectively can detect air bubbles in the fluid of a fluid system.

In an embodiment, it is an object to provide a fluid assist arrangement which can prevent air bubbles in a fluid from entering a subject when the fluid is dispensed through a fluid system.

In an embodiment, it is an object to provide a fluid assist arrangement where multiple fluid characteristics can be determined during a dispensing of a fluid in a fluid system and be controlled to ensure a safe dispensing of the fluid.

This and other objects have been solved by the invention or embodiments thereof as defined in the claims or as described herein below.

It has been found that the invention or embodiments thereof have a number of additional advantages, which will be clear to the skilled person from the following description.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

The term “substantially” should herein be taken to mean that ordinary product variances and tolerances are comprised.

The phrase “a characteristic radius of a circular or oval shape means herein a radius of a circular shape encircling a same cross sectional area as the cross sectional area encircled by the circular or oval shape.

The phrase “a characteristic diameter of a circular or oval shape means herein a diameter of a circular shape encircling a same cross sectional area as the cross sectional area encircled by the circular or oval shape.

Reference made to “some embodiments” or “an embodiment” means that a particular feature, structure, or characteristic described in connection with such embodiment(s) is included in at least one embodiment of the subject matter disclosed. Thus, the appearance of the phrases “in some embodiments” or “in an embodiment” in various places throughout the specification is not necessarily referring to the same embodiment(s). Further, the skilled person will understand that particular features, structures, or characteristics may be combined in any suitable manner within the scope of the invention as defined by the claims.

Throughout the description or claims, the singular encompasses the plural unless otherwise specified or required by the context.

All features of the inventions and embodiments of the invention as described herein including ranges and preferred ranges may be combined in various ways within the scope of the invention, unless there are specific reasons not to combine such features.

The fluid assist arrangement of the invention may advantageously be applied to assists dispensing of a fluid in a fluid system. Typically, a fluid system comprises a drip chamber and a fluid tube, where the drip chamber is in fluid connection with the fluid tube. Preferably, the fluid system further comprises a compressible bag with a fluid to be dispensed, where the compressible bag is fluidly connected to the drip chamber by a second fluid tube. The fluid entering the drip chamber will typically be in the form of fluid drops that drop into a reservoir of the fluid in the lower part of the drip chamber.

The fluid assist arrangement of the invention comprises a monitoring device. The monitoring device comprises a body having a front side and a back side. The front side of the body comprises a body recess. The body recess may define a body recess volume. The monitoring device comprises one or more light emitting elements as well as one or more light receiving elements. Both the one or more light emitting elements and the one or more light receiving elements are arranged to emit at least one light beam to pass at least a portion of the body recess volume. In an embodiment at least one of the one or more light emitting elements and the one or more light receiving elements are arranged in the body recess. The one or more light emitting elements are configured to emit at least one light beam having a beam axis.

Advantageously, the one or more light receiving elements are configured to receive at least a portion of said at least one light beam and to convert said portion of said at least one light beam into an electrical signal. Advantageously the one or more light receiving elements are arranged opposite to the one or more light emitting elements, meaning that the one or more light receiving elements respectively are arranged at the beam axis of at least one corresponding of the one or more light emitting elements. The one or more light receiving elements are configured to receive at least a portion of said at least one light beam, and advantageously configured to convert the received portion of the at least one light beam into an electrical signal.

It has been found that the one or more light emitting elements and the one or more light receiving elements provides an accurate and cost-effective monitoring of the fluid passing through the at least one light beam, and that the electrical signal can be used to determine a fluid characteristic of the fluid passing through the light beam using an assist controller.

The one or more light emitting elements are advantageously arranged in the body recess and/or in a light receiver chamber of the body recess wall and the one or more light receiving elements are advantageously arranged in the body recess and/or in a light emitter chamber of the body recess wall at a location where an upper part of a drip chamber is when the drip chamber is accommodated in the body recess. The upper part of the drip chamber shall be understood as the half, sometimes referred to as 50 percentage, of the drip chamber that forms a part of the drip chamber being closest to the top of a drip chamber.

In an embodiment, at least one of the one or more light emitting elements is arranged to emit the at least one light beam to pass through a drip chamber zone and/or at least one of the one or more light receiving elements are arranged to receive at least a portion of the at least one light beam that has passed through the drip chamber zone, wherein the drip chamber zone is a zone of the body recess volume adapted for containing a drip chamber.

It has been found that the one or more the one or more light emitting elements and one or more light receiving elements in addition may ensure a high degree of safety for a person during an infusion session, in particular where the person is able to move around and is not confined to bed, because the fluid assist arrangement via the light emitting element(s) and light receiving elements will immediately register if the person is stumbling or falling or has a leaning posture, which may indicate that the person is uncomfortable and/or dizzy, in which case a medical person, such as a nurse may immediately be alerted. This is in particular beneficial where the fluid bag is located in a portable apparatus for dispensing the fluid e.g. as described in WO19080982 and/or WO 2021/219187. Thus, in an embodiment the assist controller is configured for sending an alarm to a medical person if and when the fluid assist arrangement via the light emitting element(s) and light receiving elements register abnormalities which may indicate that the person during an infusion session is stumbling or falling or has a leaning posture, which may indicate that the person is uncomfortable and/or dizzy.

The monitoring device further comprises an assist controller in data connection with the at least one or more light receiving elements. The assist controller is adapted to receive the electrical signal from the one or more light receiving elements. The assist controller is configured for determining a fluid characteristic of the fluid from the received electrical signal. Preferably, the assist controller is configured for correlating the determined fluid characteristic with a target. The target may be a selectable target, optionally to be selected by a user on the monitoring device or from an external device in data communication with the monitoring device. In an embodiment, the assist controller is or form part of the external device. If the correlation of the target and the determined fluid characteristic is not in compliance with a threshold, such as a selected threshold, the assist controller may instruct said fluid flow device to adjust said force acting elements as it will be described further below. The threshold may be selected together with the target or independently on the monitoring device or the external device. Sometimes, and in particular for certain drugs, the target and/or the threshold may be selected from a drug library. A drug library may comprise parameters, such as targets and thresholds for specific fluids to be dispensed in a fluid system. The drug library parameters may comprise selected targets of flow rate and flow variation including thresholds. When a user chooses specific fluid to be dispensed, the user may provide a target infusion volume and infusion time. Sometimes the user weight may be provided to determine the relevant drug library parameters for the specific fluid to be dispensed.

Preferably, the assist controller is in data communication with the one or more light emitting elements. The assist controller may be configured to control the light emitting elements through the at least one light beam, where e.g. the intensity of the light beam and/or beam angle and/or the beam axis may be adjusted.

The assist controller may comprise a computer system comprising one single computer or a plurality of computers in data communication, wireless, by wire and/or via the internet.

In an embodiment, the assist controller comprises a control unit forming part of the monitoring device.

In an embodiment, the assist controller comprises a memory and the monitoring device comprises an output arrangement. The output arrangement comprises a user interface for feeding input data to the assist controller, such as input data comprising data describing a target for said fluid characteristic and optionally a threshold for said fluid characteristic target. Preferably, the assist controller is configured for storing said input data in said memory.

In an embodiment, the output arrangement comprises a user interface for outputting output data from the assist controller, such as fluid characteristic data. Preferably, the assist controller is configured for storing said output data in the memory.

The phrase “front side”is herein used to mean a forward-facing part of the body.

The phrase “back side”is herein used to mean a backward-facing part of the body.

The phrase “light” is herein used to mean electromagnetic radiation comprising visible light and invisible light, preferably comprising wavelength in the range of from 200 to 2000 nm, such as from 300 to 900 nm, such as visible light from 380 to 700 nm.

The fluid characteristic determined by the assist controller may be at least one of a velocity, a flow rate, a temperature, a turbidity, a concentration, a pH value, and/or a volume flow, preferably the at least one fluid characteristic comprises a flow fluid characteristic, such as a velocity, a flow rate and/or a volume flow and/or the at least one fluid characteristic comprises a content fluid characteristic, such as a temperature, a turbidity, a pH value, and/or a defect content, such as a bubble content and/or a crystallized matter content.

Other relevant fluid characteristics may also be determined.

Advantageously, the body recess is adapted to accommodate a drip chamber. Preferably, the body recess is also adapted for at least temporarily retaining the drip chamber.

The phrase “temporarily retain” means that the body recess is shaped to allow the drip chamber to be secured or fixed for a preselected amount of time, such as until a force detach the drip chamber from the body recess. The force may be applied by a human.

The body recess may have the shape of a semi-cylinder. The shape shall advantageously allow a drip chamber to be accommodated and at least temporarily retained in the body recess. The temporary retain may e.g. be obtained by a retaining arrangement or any protrusions in the recess body.

The inventors of the present invention have found that the arrangement of the invention is surprisingly effective in assisting the dispensing of a fluid in a fluid system. It has been found that the one or more light emitting elements and the one or more light receiving elements ensure an effective monitoring of the fluid in the drip chamber independently of the orientation of drip chamber. It has further been found that the arrangement is simple to use and may be used with practically any size of drip chamber.

Thus the fluid assist arrangement has been found to be exceptional advantageous where the compressible bag with the fluid to be dispensed is located in a portable unit, such as a portable apparatus for dispensing the fluid e.g. as described in WO19080982 and/or WO201219187.

In an embodiment, the body is made from a polymeric material or a polymer containing material. Examples of different suitable polymeric materials are Polyethylene (PE), Polypropylene (PP), Polyvinyl Chloride (PVC), Polystyrene (PS), Polyethylene Terephthalate (PET), Polyurethane (PU), Polytetrafluoroethylene (PTFE), Nylon, Acrylic (PMMA) and Silicone. The polymeric materials made be used in combination or alone. In an embodiment, the body is made from a single piece of polymeric material.

In an embodiment, the monitoring device comprises an output arrangement configured for outputting at least the fluid characteristic, wherein the output arrangement preferably comprises a visual output arrangement and/or an audio output, such as a speaker system.

The visual output arrangement preferably comprises a display unit configured to display at least the fluid characteristic. The display unit may be at least partly touch-sensitive.

In an embodiment, the output arrangement comprises at least one touch-sensitive button adjacent to the display unit, such as in a distance between 0.1 to 3 cm from display unit. The number of touch-sensitive buttons and the distance between each touch-sensitive button and the display unit may vary depending on the use case and the displayed information. Preferably, the touch-sensitive buttons are used to change between different display interfaces, fluid characteristics displayed and/or used as emergency buttons to alert preselected persons and/or used to change a fluid characteristic and/or used to start/stop a dispensing of fluid in the fluid system.

In an embodiment, the monitoring device and/or the assist controller comprises an input arrangement configured for inputting input data for determining and/or adjusting a fluid characteristic of the fluid in the fluid system. The input arrangement may comprise an audio input, such as a microphone, and/or a visual input arrangement, such as one or more touch-sensitive buttons.

In an embodiment, the fluid characteristic is at least one of velocity, flow rate, temperature, turbidity, concentration, pH, or volume flow. Velocity is often determined as meter per second, where flow rate is typically determined as milliliter per hour or cubic meter per second. Flow rate is sometimes also referred to as drops per minute or drip factor (gtts/mL).

In an embodiment, the body recess is at least partly delimited by the body. Preferably, the body recess is defined by the body. The length of the body recess is measured along the extension of the body recess. Typically, the body recess extends from one side of the body to the opposite side of the body.

In an embodiment, the body recess is a longitudinal body recess and wherein the body has a maximal length parallel to the length of the body recess, which maximal body length is substantially equal to the length of the body recess.

In an embodiment, the length, such as the maximal length of the body is shorter than the length of the drip chamber. The length of the body is measured in the same direction as the extension length of the body recess. In an embodiment, the length of the body is shorter than 6 cm, such as shorter than 5 cm.

Advantageously, the length of the body is equal to the length of the body recess, i.e. the body recess extends across the whole body length.

The body recess has a body recess axis. Preferably, the body recess axis is centered (i.e. a center body recess) and/or parallel with the length of body recess being the extension direction of the body recess.

In an embodiment, the monitoring device comprises a retaining arrangement comprising at least one retaining element arranged in the body recess. Preferably, the at least one retaining element protrudes into the body recess, so that one part of the respective retaining element is closer to the body recess axis than a second part of the respective retaining element.

It has been found that the retaining arrangement is surprisingly effective for retaining a drip chamber, as the retaining arrangement allow the body recess to have a size that fits to many different drip chamber sizes, while the retaining arrangement provides a fixation of the drip chamber independently of its size, and at the same time the retaining arrangement provides a snap fit of the drip chamber in the body recess. Hence, in a desired embodiment, the fluid assist arrangement comprises a retaining arrangement.

In an embodiment, the body comprises a body recess wall defining the body recess and wherein the retaining arrangement is located at the body recess wall, preferably the retaining arrangement comprising the at least one retaining element comprises a protrusion which protrudes into the body recess.

In an embodiment, the retaining element is made from a resilient polymer. Advantageously, the retaining arrangement is detachably connected to the body recess, so it can be changed e.g. if the retaining arrangement is worn out. Preferably, the retaining arrangement is detachably connected to the body recess wall.

The at least one retaining element may for example be at least partially made from a resilient polymer, such as a polymer having a modulus of resilience Uᵣ of at least about 0.3 MPa, such as at least about 0.6 MPa, such as at least about 1 MPa determined according Yield strength/2*Youngs modulus, determined according to ISO 527-1:2019.

The at least one retaining element is may in an embodiment be detachably connected to the body recess wall. Thereby the least one retaining element may be replaceable e.g. for being replaced by another retaining element, such as a retaining element having a different size e.g. for use together with a different drip chamber.

In an embodiment, the retaining element comprises a force-generating element. The force-generating element is preferably an actuator and/or a spring that is adapted to engage with the drip chamber and retain the drip chamber by applying a force to the retaining element. In an embodiment, the force-generating element engages with the drip chamber through the retaining element and is arranged between the retaining element and the body, i.e. within the body recess wall.

In an embodiment, the at least one retaining element comprises at least one resilient arm adapted to engage with the drip chamber, wherein said at least one resilient arm is adapted for at least partially embrace the drip chamber to hold the drip chamber in temporally fixed condition in the body recess.

In an embodiment, the retaining element comprises one or more openings, wherein the one or more openings are arranged to allow the one or more light emitting elements to emit the at least one light beam to pass through the one or more openings. Advantageously, the one or more openings is arranged to allow the one or more light receiving elements to receive at least a portion of the at least one light beam that has passed through the one or more openings. It has been found that when the retaining element comprises one or more openings arranged to allow the one or more light emitting elements to emit the at least one light beam through the one or more openings a very precise and accurate fluid characteristic of the fluid may be obtained.

Preferably, the one or more light receiving elements are arranged opposite to the one or more light emitting elements. In an embodiment, the retaining element comprises at least a first opening and a second opening, wherein the first opening is arranged to allow the one or more light emitting elements to emit at least one light beam to pass through the first opening to pass through the drip chamber zone, and wherein the second opening is arranged to allow the one or more light receiving elements to receive at least a portion of the at least one light beam that has passed the drip chamber zone and passed through the second opening.

In an embodiment, at least one of the one or more light emitting elements is located in a light emitter chamber of the body recess wall and wherein the body has a light passage between the light emitter chamber and the body recess volume, preferably the drip chamber zone of the body recess volume to provide that the one or more light emitting elements are adapted for emit the at least one light beam into the body recess volume, preferably into the drip chamber zone.

In an embodiment, at least one of the one or more light receiving elements are located in a light receiving chamber of the body recess wall and wherein the body has a light passage between the light receiver chamber and the body recess volume, preferably the drip chamber zone of the body recess volume to provide that the one or more light receiving elements are adapted for receiving at least a portion of the at least one light beam that has passes through the body recess volume, preferably passes through the drip chamber zone.

Advantageously, the one or more light emitting elements is located to emit the at least one light beam to pass through the body recess volume, preferably to pass through the drip chamber zone of the body recess volume and wherein said one or more light receiving elements are located to receive at least a portion of said at least one light beam that has passed through the body recess volume, preferably that has passed through the drip chamber zone of the body recess volume.

Advantageously, the one or more light emitting elements is located to emit the at least one light beam to pass through the body recess volume, preferably to pass through the drip chamber zone of the body recess volume, wherein the beam center axis crosses the body recess axis with an angle of from 65°, to 115°, such as from 75°, to 105°, such as from 75°, to 110°, such as from 85°, to 95°, such as orthogonally. In an embodiment, each of the at least one retaining element independently of each other has a length parallel to the body recess axis and wherein the length of the at least one retaining element is 2 % to 100 %,, such as from 5 % to 50 % of the length of the body recess, such as at least ⅓ of the length of the body recess, such as at least ⅔ of the length of the body recess, such as substantially equal to the length of the body recess.

In an embodiment, the length of the retaining element is equal to at least ⅓ of the length of the body recess, such as at least ⅔ of the length of the recess, such as substantially equal to the length of the recess. ⅓ is the same as 33.34 percentage or sometimes referred to as one-third.

Preferably, at least a part of the retaining element protrudes from the body recess wall towards the body recess axis.

In an embodiment, the retaining arrangement comprises at least two retaining elements, preferably arranged opposite to each other. It has been found that a plurality of retaining elements may provide a better retainment of some drip chambers when accommodated in the body recess.

The body recess has a surface, which may be rough, smooth or a combination. In an embodiment, the wall of the body recess forming the surface of the body recess may conveniently be at least partly made from a resilient polymer.

In an embodiment, the body recess is curved, preferably the body recess wall is curved.

In an embodiment, the body recess is curved from 160° to 200°, such as from 170° to 190°, such as from 175° to 185°, such about 180° around the body recess axis. Preferably the body recess wall is curved to form a channel shape around the body recess axis for hosting the drip chamber.

In an embodiment, the monitoring device comprises at least two light emitting elements, such as at least three, each configured for emitting respective light beams. Preferably, the beam axes of the at least two light beams are parallel. In an embodiment, the beam axes of at least two of the light beams intersect each other at least partly.

In an embodiment, the one or more light emitting elements is one or more light emitting diodes emitting at least one light beam. In an embodiment, the one or more light emitting elements is a laser source emitting a laser beam. If there is more than one light emitting element then there may be two or more light emitting diodes, two or more laser sources, or a combination of both light emitting diodes and laser sources. Preferably, the light emitting diode emits light within the visible spectrum from 380 to 700 nm. In an embodiment, the light emitting diode emits light within the infrared field, preferably within the near infrared field from above 700 to 2500 nm. Where there are two or more light emitting elements these may conveniently emit at different wavelengths.

Advantageously, the at least one light beam illuminates a wide field so as to cover most of a plane. Preferably, the at least one light beam illuminates more than 50% of a perpendicular plane to the body recess axis in the body recess, such as of the body recess volume, such as of the drip chamber zone of the body recess in a plane perpendicular to the body recess axis,. In an embodiment, the at least one light beam illuminates more than 60% of the perpendicular plane, such as more than 65%. Preferably the at least one light beam illuminates more than 60%, such as more than 65% of the body recess in a plane perpendicular to the body recess axis. In an embodiment, the at least one light beam illuminates more than 50% of the body recess, such as of the body recess volume, such as of the drip chamber zone of the body recess in a horizontal plane of the body recess wall.

In an embodiment, the monitoring device comprises at least two light emitting elements, and wherein the at least two light emitting elements are arranged opposite to one another.

Advantageously, opposite arranged light emitting elements provides a more efficient coverage of a plane perpendicular to the body recess axis, as most light beams have a beam angle that disperses the light.

In an embodiment, the at least one light beam has a beam angle larger than 10° and smaller than 100°, such as larger than 15° and smaller than 50°.

The body recess wall may comprise a plurality of body recess sections defining the body recess. In an embodiment, the body recess wall comprises two opposite body recess sections.

In an embodiment, the one or more light emitting elements and the one or more light receiving elements are arranged vertically above each other or beside each other or spaced apart.

The terms “above” and “beside” are herein used to define a spatial orientation between features. “above” refers to a relationship between features being above each other in a vertical direction when the body recess and/or the recess axis is in a vertical orientation. “beside” refers to a relationship between features being beside each other, sometimes referred to as next to each other, in a horizontal direction when the body recess and/or the recess axis is in a vertical orientation.

Each light beam may independently of each other have a selected wavelength range. For example where there are two or more the one or more light emitting elements, these two or more the one or more light emitting elements may conveniently be configured for emitting light beams having different wavelengths, Thereby the at least one fluid characteristic may be determined with an even higher accuracy.

In an embodiment, the one or more light emitting elements and the one or more light receiving elements are arranged on one side of the body recess wall, and wherein a reflecting element is arranged opposite to the one or more light emitting elements to reflect the at least one emitted light beam to the one or more light receiving elements.

In an embodiment, the monitoring device comprises at least two light emitting elements and at least two light receiving elements, and wherein a first pair comprising at least one of the at least two light emitting elements and at least one of the at least two light receiving elements is arranged opposite to each other in a first plane perpendicular to the body recess axis, and wherein a second pair comprising at least one other of the at least two light emitting elements and at least one other of the at least two light receiving elements is arranged opposite to each other in a second plane perpendicular to the body recess axis. Advantageously, the first and second perpendicular planes do not intersect.

In an embodiment, the monitoring device comprises at least one optical filter located to filter the light beam between the one or more light emitting elements and said one or more light receiving elements. The at least one optical filter may conveniently comprise at least one of a band-pass filter, a long-pass filter, a short-pass filter, a dichroic filter and/or a polarized filter. By applying an optical filter otherwise disturbing reflections e.g. from a surface of a fluid in the drip chamber, and/or from indecent light.

The at least one filter may be located in or forming part of the light emitter chamber of the body recess wall and/or the at least one filter may be located in the body recess at a location between a body recess wall and the drip chamber zone.

In an embodiment, the fluid assist arrangement comprises an orientation sensor, such as a gyroscope and or a magnetometer adapted for determining tilt of the fluid assist arrangement.

In an embodiment, the fluid assist arrangement comprises a gyroscope. Preferably, the gyroscope is adapted for measuring rotational rate, and can detect if the fluid assist arrangement is in an upright position, a flat position, and/or in motion.

In an embodiment, the fluid assist arrangement comprises a fluid flow device, such as at least one fluid flow device for adjusting a liquid flow in the fluid tube, wherein the fluid assist arrangement. The fluid flow device comprises one or more force acting elements, wherein the one or more force acting elements are configured for adjustably constricting the fluid tube to adjust fluid flow. It has been found that constricting the tube using force acting elements is a cost effective and efficient way of controlling the velocity and/or the flow rate of the fluid dispensed in the fluid system.

In an embodiment, the fluid flow device comprises a tube element adapted to accommodate a tube, and wherein the one or more force acting elements are arranged at one or more sub-recesses in a tube recess wall having a passage into the tube recess.

The tube recess has a length along a tube recess center axis.

In an embodiment, the tube recess is defined by a tube recess wall. The tube recess wall advantageously comprises at least one sub-recess, wherein the at least one sub-recess comprises a recess passage into the tube recess.

Preferably, each of the one or more force acting elements comprise a constriction member connected to an extending member, and wherein the constriction member is adapted to engage with the fluid tube.

Preferably, at least a part of the extending member is located in the at least one sub-recess of the tube recess and wherein at least a portion of the constriction member is adapted to be located in the tube recess when in operation to constrict the fluid tube, wherein the force acting elements is configured for extend the extending member to pass at least the portion of the constriction member through said recess passage into said tube recess to constrict said fluid tube

Advantageously, the constriction member is rotatably connected to the extending member, which has been found to be a flexible and useful way of allowing the constriction member to adapt the shape of the fluid tube.

In an embodiment, the extending member is configured to extend and to retract. Advantageously, the extending member is an actuator or acts as an actuator, such as an actuator controlled by a step motor.

In an embodiment, the fluid flow device comprises at least one threaded element extending along a length of the fluid flow device, and wherein the extending member of each of the one or more force acting element is connected to the threaded element and adapted to move along the length of the threaded element when the threaded element is rotating.

The threaded member may conveniently located in the at least one sub-recess, wherein the at least one sub-recess comprises a longitudinal sub-recess having a length parallel to a length of the tube recess, such as parallel to the tube recess center axis and wherein the length of the sub-recess preferably is at least 25 % of the length of the tube recess, such as wherein the length of the sub-recess is at least 40 %, such as at least 60 %, such as at least 80 %, such as at least 90% of the length of the tube recess. It has been found that using a threaded member in the operation of the force acting elements, a highly accurate control and very fast adjustment of the fluid flow may be obtained.

In an embodiment, the one or more force acting elements are adapted to move along the length of the tube recess. The one or more force acting elements may move based on an electric signal, preferably the electrical signal is from the assist controller.

In an embodiment, the constriction member comprises a plurality of elongate parts, optionally at least one end of each of the plurality of elongate parts is connected to an end of one of the other elongate parts.

The motions of the one or more force acting elements may be controlled by a method comprising submitting at least one electric signal, preferably the assist controller is adapted for controlling the motions of the one or more force acting elements e.g. by submitting the electrical signal.

In an embodiment, the at least one fluid flow device comprises a main housing and a lid part connected to the main housing, and wherein the lid part at least partly covers the fluid tube when the fluid tube is placed in the main housing of the at least one fluid flow device.

The main housing is preferably at least partly provided by said tube recess. In an embodiment, the main housing is formed by the tube recess.

Advantageously, the tube recess is a recess from the back side of the body, i.e. the tube recess is located at the back side of the body. By locating the tube recess at the back side of the body the fluid assist device may be very compact and maintain an excellent insight of all the functions of the fluid assist arrangement.

The fluid flow device may have a lid part connected to the fluid flow device. The lid part covers at least partly the fluid tube when the fluid tube is placed in the fluid flow device. Preferably, the lid part is pivotably connected to the remaining part of the fluid flow device.

In an embodiment, the one or more force acting elements of the fluid flow device comprises at least a pair of force acting elements arranged opposite to each other. Preferably, the fluid flow device comprises at least two pairs of force acting elements, and wherein each pair of force acting elements are independently operable. Advantageously, each of the force acting elements of a pair of force acting elements are opposite acting, preferably such that they are moving mirrored to each other.

In an embodiment, the fluid flow device is configured to control fluid flow based on the determined fluid characteristic.

In an embodiment, the fluid flow device comprises a protrusion member arranged in a selected tube distance from the one or more force acting elements. Preferably, the tube distance is selected based on the diameter of the tube, and wherein the one or more force acting elements is adapted to constrict the fluid tube by moving towards the protrusion member.

In an embodiment, he one or more force acting elements of the at least one fluid flow device comprises at least a pair of force acting elements, wherein one of the a pair of force acting elements comprises a protrusion member arranged to protrude in a selected tube distance from the other one of the pair of force acting elements. The protrusion member may act as a blind force acting element of the pair of force acting elements since it is stationary to form a contra force.

The protrusion member has a length and a width. In a desired embodiment, the length of the protrusion member is longer than the width and/or the selected tube distance of the protrusion member. The width may be identical to the protrusion direction. Preferably the length of the protrusion member being at least substantially parallel with the body recess. In another desired embodiment, the width of the protrusion member is longer or similar to the length of the protrusion member.

In an embodiment, the fluid assist arrangement comprises an air detector. Preferably, the air detector comprises an acoustic sensing element adapted to detect air in the fluid tube. The air detector may use acoustic waves to identify changes in the fluid, such as air and/or air bubbles in the fluid. Detection of air and/or air bubbles may cause the fluid flow device to constrict the fluid tube to prevent the detected air and/or air bubbles from being dispensed through the fluid system. The air detector is preferably in data connection with the assist controller. It has been found that using acoustic waves for detecting air and/or air bubbles in the fluid is very precise and reliable.

In an embodiment, the air detector is arranged adjacent to the fluid flow device. Preferably, the fluid flow device comprises the air detector.

In an embodiment, the fluid assist arrangement comprises a crystallized matter detector configured for detection crystals that may have formed in the fluid. If crystals are detected, the assist controller may conveniently cause the fluid flow device to constrict the fluid tube to prevent the detected crystals from being dispensed through the fluid system.

The assist controller may be configured for controlling said one or more force acting elements at least partially in dependence of the air detector and/or at least partially in dependence of the air detector and/or at least partially in dependence of the crystallized matter detector. Preferably, the assist controller is configured for submitting an electrical signal to the at least one fluid flow device to provide the of the one or more force acting elements to constricting the fluid tube to terminate the fluid flow in the fluid tube at least temporally, preferably the assist controller is configured for simultaneously providing an alarm.

In an embodiment, the fluid flow device is connected to the monitoring device. Preferably, the fluid flow device is connected to the body of the monitoring device.

In an embodiment, the lid part covers the one or more force acting elements and the air detector.

In an embodiment, the fluid flow device and/or the air detector are arranged beside and/or spaced apart from the body recess.

In an embodiment, the fluid flow device is located with access from the backside of the body.

In an embodiment, the fluid assist arrangement comprises a monitoring device and an air detector, and preferably an assist controller.

In an embodiment, the fluid assist arrangement comprises a fluid flow device, and preferably an assist controller. The fluid flow device and the assist controller may be as detailed above, but without the monitoring device.

In an embodiment, the fluid assist arrangement comprises a fluid flow device and an air detector, and preferably an assist controller.

Preferably, the fluid flow device is arranged to have the fluid tube located in vertical orientation, such that the fluid in the fluid tube is to flow downwards from a top position of the fluid recess and downwards. Thereby the risk of air bubbles to be injected to an individual under infusion treatment may be further reduced and in practice eliminated. The body may advantageously comprise at least one tube guide structure for guiding a tube section of the fluid tube from an exit of the drip chamber and upwards to an inlet end of the at least one fluid flow device.

In an embodiment, the at least one fluid flow device is arranged to have the length of the tube recess horizontally orientated. It has been found that by orientating the fluid flow device horizontally, the risk of air bubbles being injected to an individual under infusion treatment is may be further reduced and in practice eliminated. At the same time there is no risk of orientating the fluid tube incorrectly in the tube recess.

In an embodiment, the fluid assist arrangement comprises two or more fluid flow devices, such as two or three fluid flow devices. For example a first fluid flow device may comprise one or more force acting elements adapted for reducing the flow in the fluid tube by constricting the fluid tube to a desired level, a second fluid flow device may comprise a bubble detector and/or a crystallized matter detector and a third fluid flow device may comprise one or more force acting elements adapted for pumping the fluid in the fluid tube forward to be dispensed to an individual. The first fluid flow device may conveniently be a vertical fluid flow device, where the fluid tube is arranged in the vertical tube recess with an upwards flow direction. Thereby the flow may very fast be slowed down or even stopped, e.g. in a bubble or crystal is detected in the second fluid flow device. The second fluid flow device may conveniently be horizontally arranged. The third fluid flow device may conveniently be vertically arranged with downwards flow direction to ensure a desired high velocity, now that the fluid has been inspected for bubbles and/or crystallized matter. Alternatively, where the fluid is of a type that should be dispensed relatively slow the third fluid flow device may be vertically arranged with an upwards flow direction or the third fluid flow device may be horizontally arranged or any orientation there between.

In an embodiment, the fluid assist arrangement comprises at least one tube guide structure for guiding the a tube section of the fluid tube from an exit of the drip chamber to an inlet end of the at least one fluid flow device and/or between fluid flow devices, wherein the at least one tube guide structure comprises a channel recess section in the body and/or at least one tube guide loop. The at least one tube guide structure is arrange for supporting the fluid tube close to the monitoring device and/or close to the fluid flow device.

The at least one tube guide structure reduces the risk that the fluid tube may be tangled in other equipment, such as another fluid tube. In addition, the at least one tube guide structure may ensure that the tube is correctly guided through the one or more fluid flow device.

In an embodiment the fluid assist arrangement comprises at least one time-of flight (TOF) sensor.

TOF sensors are well known sensor within the radar technology. Heretofore, it has never been considered applying TOF sensors in medical devices.

The at least one TOF sensor may preferably comprise a first TOF sensor, wherein the first TOF sensor is located to pointing downwards from at least one of the monitoring device and/or from the fluid flow device when the fluid assist arrangement is in use. Preferably the first TOF sensor is adapted for determining a height location of the fluid assist arrangement.

It has been found that when determining the height location of the fluid assist arrangement, such as the height location relative to a floor, to a bed or to a reference element e.g. a reference element located at a height corresponding to the infusion site of the individual subjected to the IV infusion, the height in the liquid column in the fluid tube may be determined and there the pressure at the infusion site may be determined as well. This information may for example be applied for setting the fluid velocity.

In an embodiment, the monitoring device has a downwards facing edge portion adapted to face downwards when the fluid assist arrangement is in use. The at least one TOF sensor comprises a TOF sensor, preferably said first TOF sensor located in or at said downwards facing edge portion and/or located at a surface region of said downwards facing edge portion.

The fluid assist arrangement may comprise a second TOF sensor, wherein the second TOF sensor is located to pointing upwards from at least one of the monitoring device and/or from the fluid flow device when the fluid assist arrangement is in use. Preferably the second TOF sensor is adapted for determining a distance to an upwards located element and/or marker.

Thereby an even more accurate location of the fluid assist arrangement and thereby of the drip chamber may be determined.

In an embodiment, the monitoring device has an upwards facing edge portion adapted to face upwards when the fluid assist arrangement is in use. Preferably the second TOF sensor, located in or at said upwards facing edge portion and/or located at a surface region of said upwards facing edge portion.

The at least one TOF sensor conveniently comprises an emitter and a receiver, wherein the emitter is configured for emitting a TOF light beam and wherein the receiver is adapted to receive at least a portion of the TOF light beam reflected from a surface onto which the emitted light beam is impinging.

In an embodiment, the at least one TOF sensor comprises at least one indirect TOF (iTOF) sensor and/or at least one direct TOF (dTOF) sensor. Preferably the at least one iTOF sensor comprises a phase shift TOF. The at least one phase shift TOF is configured for measure the phase shift of a light wave reflected from as surface. The at least one dTOF sensor is a pulsed TOF sensor, preferably having a modulation frequency of from 1 MHz to 100 MHz, such as up to 30 MHz.

In an embodiment, the at least one TOF sensor comprises at least one of a continuous wave (CW) TOF sensor, a phase-shift TOF sensor, a pulsed TOF sensor, a flash TOF sensor, a line scan TOF sensor and/or any combinations thereof.

In an embodiment, the at least one TOF sensor comprises a continuous wave modulated TOF sensor wherein the continuous wave modulated TOF sensor is configured for measure the phase difference between the sent and received signals. Different shapes of signals are possible, e.g., sinusoidal, square waves, etc. Cross-correlation between the detected and emitted signals allows a very accurate phase estimation, which is directly related to distance if the modulation frequency of the emitted signal is known.

In an embodiment, the at least one TOF sensor comprises a pulsed-modulated TOF sensor. The pulsed-modulated TOF sensor measures the time-of-flight directly. Advantageously, pulses are relatively short, such as with a pulse width of up the 100 ns, advantageously, in the range of from 1 to 50 ns, preferably having a fast rise-and fall-times.

It has been found that especially modulated single pixel TOFs may have a high accuracy, even where the distance is relative low, such as 5 cm or less, such as 2 cm or less, such as 1 cm or less or even a few mm or less.

The at least one TOF sensor is advantageously configured for operating in a relatively short range, such as within a distance of up to about 150 cm, such as up 100 cm, such as up to 50 cm, such as up to 30 cm.

The emitter of the at least one TOF sensor may comprise at least one of a light emitting diode (LED) and a laser diode, such as a laser diode selected from an edge-emitting lase and a vertical-cavity surface-emitting laser (VCSEL), preferably the emitter of the at least one TOF sensor comprises at least one IR LED, preferably operating in the wavelength range in the near-infrared spectrum from 700 nm to 1400 nm, preferably from 850 nm to 940 nm.

IR light source operating with a center wavelength of about 900 nm or larger may be preferred for preventing crosstalk with ambient light.

Infrared LEDs are generally more cost-effective than laser diodes and may in addition be simple to integrate. For longer range Laser diodes may be preferred, however for the relative short range required for the at least one TOF sensor, the LEDs have found to be very effective. In addition, LEDs are eye safe, which is a large benefit

Since wavelengths in the mid-IR range (1400-3000) and especially in the far IR (3000 nm-1 mm) may generate large amounts of heat. Therefore if using a light source comprising mid-IR of far-IR wavelength, the heat generation may advantageously be controlled.

The at least one TOF sensor comprises a few pixel TOF sensor and/or a line-scan TOF sensor, such as a pixel TOF sensor that comprises up to 1024 pixels, such as up to 256 pixels, such as up to 64 pixels, e.g. 8, 4 or 1 pixel(s).

Advantageously, the at least one TOF sensor comprises a single pixel sensor. Preferably the at least one single pixel sensor comprises a pulsed TOF sensor and/or a continuous wave modulated TOF sensor, preferably the TOF sensor is operating at a modulation frequency of from 1 MHz to 100 MHz, such as up to 30 MHz.

The fluid assist arrangement according to any one of the claims, wherein the fluid assist arrangement is configured for being in data communication with at least one other fluid assist arrangement and/or with a central controller.

This embodiment is in particular preferred where an individual are to be treated with two or more infusion fluids simultaneously or overlapping in time. Certain patients, such as cancer patients may have two or more infusion treatments simultaneously or overlapping. Here it is often important to control the relative flow of the respective fluids into the vein of the patient. Therefore where two or more fluids are to be infused simultaneously or overlapping in time using respective fluid assist arrangement, it is highly advantageously that the fluid assist arrangement may be adjusted relative to each other. This may be provided by providing that the fluid assist arrangement is configured for being in data communication with at least one other fluid assist arrangement and/or with a central controller.

Preferably said fluid assist arrangement is configured for being in data communication with the at least one other fluid assist arrangement and/or with the central controller by a wired connection and/or by a wireless connection.

Generally it is preferred that the assist controller of the respective fluid assist arrangements are in communication with the central controller and that the two or more fluid assist arrangement are configured for communicate via the central controller and/or being adjustable by the respective assist controller based on data transmitted from the central controller to thereby ensuring that the two or more fluid assist arrangement may be adjusted relative to each other.

In an embodiment, the fluid assist arrangement comprises transmitter and/or a transceiver, wherein said transmitter and/or a transceiver comprises a radio-frequency sensor, such as a chip sensor (RFID-active or passive), a Bluetooth sensor or any combinations thereof preferably suitable for being in data communication with at least one fluid assist arrangement and/or with the central controller.

Advantageously, each fluid assist arrangement comprises a unique code. Thereby the central controller may be configured for recognize each respective fluid assist arrangement.

The invention also comprises the fluid assist arrangement in combination with a drip chamber. The fluid assist arrangement may be as described above. The drip chamber may be as described above.

The body recess of the fluid assist arrangement is adapted for receiving and temporally retain the drip chamber in the body recess, whereby the drip chamber preferably defines a drip chamber zone of the body recess when mounted in the body recess.

The body recess has a body recess axis and a body recess radius determined as the minimum distance between the body recess wall and the body recess axis. The drip chamber may preferably have a characteristic maximal outer radius which is less than the body recess radius, such as at least 0.2 cm less, such as at least 0.1 cm less, such as at least 1 cm less than the characteristic maximal outer radius.

The invention also comprises a fluid assist installation. The fluid assist installation comprises a plurality of fluid dispensing module, wherein each fluid dispensing module comprises a fluid assist arrangement as described above.

Advantageously, the fluid assist arrangement of one or more of the plurality of fluid dispensing modules is in data communication with the fluid assist arrangement of at least one other of the plurality of fluid dispensing modules and/or is in data communication with a central controller. Preferably the fluid assist arrangement of each of the plurality of fluid dispensing modules is in data communication with the fluid assist arrangement of at least one other of the plurality of fluid dispensing modules and/or is in data communication with the central controller.

Advantageously, each of the plurality of fluid dispensing modules comprises an IV bag and a fluid system comprising a drip chamber and a fluid tube in fluid connection with the drip chamber and wherein the fluid assist arrangement is arranged for assisting dispensing of a fluid from the IV bag via the fluid system, wherein the drip chamber is at least partly located in the body recess of said fluid assist arrangement.

In an embodiment, the IV bag of each of the one or more of said plurality of fluid dispensing modules is hanging at an IV pole and/or wherein the IV bag of each of the one or more of said plurality of fluid dispensing modules is located in a dispensing pump, such as a dispensing pump as described in WO19080982 and/or WO201219187.

Advantageously, the fluid assist arrangement of one or more of the plurality of fluid dispensing modules is in wired data communication and/or in wireless data communication with the fluid assist of at least one other of the plurality of fluid dispensing modules and/or is in data communication with central controller, e.g. as described above.

The invention also comprises a flow device arrangement for assisting dispensing of a fluid in a fluid system comprising a fluid tube, wherein the arrangement comprises an assist controller and a fluid flow device.

The fluid flow device comprises a main housing comprising a tube element, such as a tube recess, adapted to accommodate a fluid tube, and preferably a lid part pivotably connected to the main housing, and one or more force acting elements arranged at the tube element.

The one or more force acting elements are configured for adjustably constricting the fluid tube to adjust a fluid flow in the fluid tube preferably based on an electrical signal from an assist controller. The force acting elements may be as described above.

The flow device arrangement may advantageously comprise an air detector and/or a crystallized matter detector arranged at the tube element, wherein the air detector comprises an acoustic sensing element adapted to detect air in the fluid tube and the crystallized matter detector comprises an optical detector The flow device arrangement, may preferably form part of a fluid assist arrangement as described above and claimed herein.

### BRIEF DESCRIPTION OF THE EXAMPLES AND DRAWING

In the following, the invention will be further illustrated by the description of a number of illustrative and non-limiting embodiments and parts thereof of the present invention, with reference to the appended figures.

The figures are schematic and are not drawn to scale and may be simplified for clarity. Throughout, the same reference numerals are used for identical or corresponding parts.

FIG. 1 illustrates a fluid assist arrangement of an embodiment of the invention.

FIG. 2 illustrates a fluid flow arrangement of an embodiment of the invention with a drip chamber retained in the body recess.

FIG. 3 illustrates a variation of the embodiment shown in FIG. 2.

FIG. 4a illustrates a fluid flow arrangement of an embodiment of the invention seen from a top side of the body.

FIG. 4b illustrates a fluid flow arrangement of another embodiment of the invention seen from a top side of the body.

FIG. 5 illustrates a fluid flow arrangement of an embodiment of the invention with a fluid flow device.

FIGS. 6A and 6B illustrate variations of a fluid flow device and an air detector of embodiments of the invention comprising where the fluid flow device comprises an air detector.

FIG. 7 illustrates a fluid assist arrangement of an embodiment of the invention with a fluid flow device and an assist controller in data communication with the arrangement.

FIGS. 8A and 8B, respectively illustrates a fluid flow device of an embodiment of the invention comprising one respectively a plurality of force acting elements.

FIGS. 9A-9D illustrate an extending member and a force acting member of a fluid flow device of an embodiment of the invention, where the force acting member is shown at different vertical locations.

FIGS. 10A-10F illustrate variations of the embodiments shown in FIG. 8A and FIG. 8B.

FIGS. 11A-11N are schematic views of light emitting elements of embodiments of the invention.

FIGS. 12A and 12B are schematic views of light beams emitted by the one or more the one or more light emitting elements of embodiments of the invention.

FIGS. 13A and 13B illustrate variations of the embodiment shown in FIG. 1.

FIGS. 14A and 14B illustrate an arrangement of an embodiment of the invention comprising a monitoring device, a fluid flow device and an air detector.

FIG. 15 illustrates from a top view an arrangement of an embodiment of the invention.

FIG. 16 illustrates a fluid assist arrangement of an embodiment of the invention with a monitoring device and a fluid flow device, where the monitoring device is without a display unit.

FIG. 17 illustrates a fluid assist arrangement of an embodiment of the invention with a fluid flow device.

FIG. 18 illustrates different drip chambers suitable for use with the invention.

FIGS. 19A and 19B show a fluid assist arrangement of an embodiment of the invention.

FIG. 20 shows a fluid assist arrangement 32 of an embodiment of the invention seem from the front side.

FIG. 21 shows a fluid assist arrangement 32 of another embodiment of the invention seen in a top view

FIG. 22 shows a variation of the fluid assist arrangement 32 FIG. 21, wherein the fluid assist arrangement comprises two fluid flow devices 20a, 20b.

FIG. 23 shows a variation of the fluid assist arrangement 32 FIG. 20.

FIG. 24A illustrates a fluid assist arrangement of an embodiment of the invention where the retaining element is in the form of an elastic band 14d.

FIG. 24B illustrates a portion of the fluid assist arrangement of FIG. 14A.

FIG. 25 illustrates a fluid assist arrangement of an embodiment of the invention comprising at least one TOF sensor.

FIG. 26 illustrates a fluid assist installation comprising a fluid assist arrangement 39a of an embodiment of the invention.

FIG. 27 illustrates a fluid assist installation of an embodiment of the invention.

FIG. 28 illustrates another fluid assist installation of an embodiment of the invention.

FIG. 29 illustrates yet another fluid assist installation of an embodiment of the invention.

FIG. 30 illustrates a fluid assist installation of an embodiment of the invention, comprising a rack of fluid assist arrangements.

FIG. 31 illustrates a fluid assist installation of another embodiment of the invention, comprising a rack comprising one or more fluid assist arrangements.

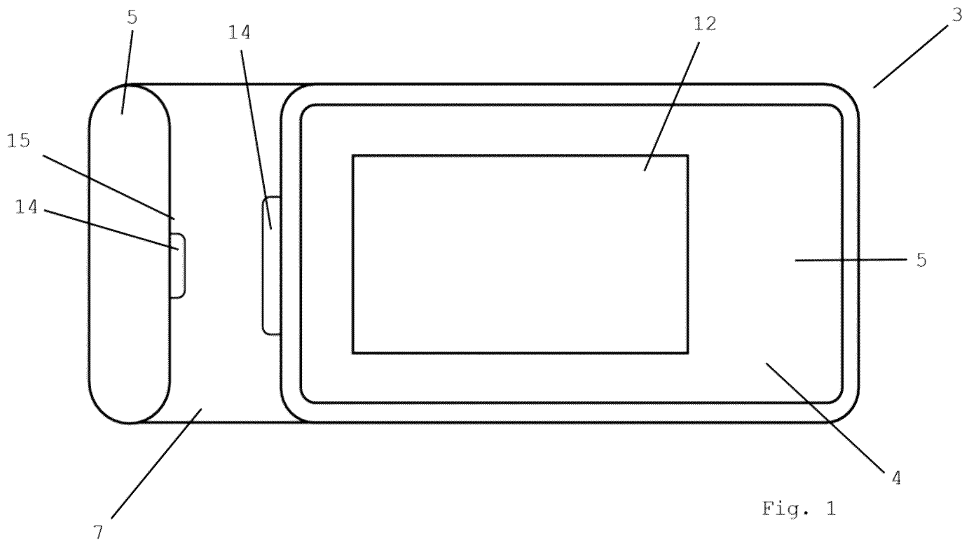

FIG. 1 shows a fluid assist arrangement comprising a monitoring device 3. The monitoring device comprises a body 4 with a front side 5, a back side 6 (not shown) and a body recess 7 arranged in the front side. The front side 5 is divided into two sections by the body recess 7. The monitoring device shown in FIG. 1 also comprises an output arrangement in the form of a display unit 12 arranged in the front side 5 of the body. The display is preferably in data connection to an assist controller arranged within the body of the monitoring device (not shown).

The body recess 7 comprises a retaining arrangement comprising two retaining elements 14 arranged opposite to each other and protruding from the body recess wall 15. The retaining elements in this embodiment are different in size. The shapes of the retaining elements 14 are adapted to retain a drip chamber in temporally fixed condition when the drip chamber is arranged in the body recess.

In FIG. 2, the fluid assist arrangement comprising a monitoring device is shown with a drip chamber 1 arranged within the body recess 7. The shown drip chamber 1 is longer than the body recess 7 and thereby extends outside in both ends of the body recess. The drip chamber 1 is retained by two retaining elements 14 arranged opposite to each other. The drip chamber is fluidically connected to a first fluid tube 2a at the top of the drip chamber 1 and a second fluid tube 2b at the bottom of the drip chamber 1. A light emitting element 8 is arranged above one of the retaining elements 14 and a light receiving element 9 is arranged opposite to the light emitting element 8 and above the second retaining element 14. The light emitting element is arranged to allow the light beam (not shown) emitted to propagate through the drip chamber 1 also referred to as the drip chamber zone of the body recess. The light receiving element 9 is arranged to receive at least a portion of the emitted light beam emitted from the light emitting element 8, passing through the drip chamber zone and then received by the light receiving element. As described above, the light received by the one or more light receiving element mat then be applied for determine at least one fluid characteristic.

The monitoring device shown in FIG. 2 comprises a plurality of touch-sensitive buttons 13. In this embodiment, the touch-sensitive buttons 13 are shown as a plus, as a minus, as an alarm, as a drop, and as a power on/off touch-sensitive button 13. The skilled person will understand that other or alternative touch-sensitive buttons may be applied and that the touch-sensitive buttons 13 may be selected based on the application of the fluid assist arrangement. The buttons may for example be used for inputting a target fluid characteristic or for outputting a determined fluid characteristic.

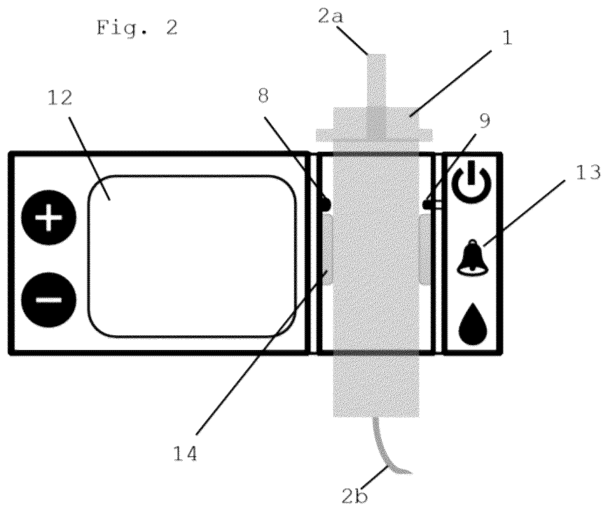

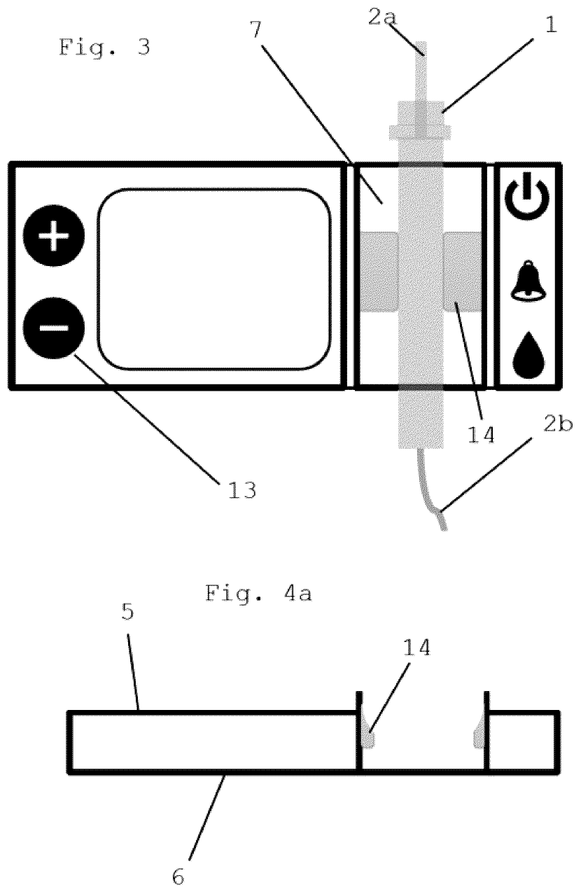

FIG. 3 shows the fluid assist arrangement of the embodiment of FIG. 2, where a drip chamber 1 with a smaller diameter is shown. As it can be seen the retaining elements 14 of FIG. 3 is protruding further out from the body recess wall 15 compared to the retaining elements 14 in FIG. 2, as the drip chamber 1 has a smaller diameter. Thus, fluid assist arrangement of embodiments of the invention may be applied with many different shapes and sizes, because the body recess 7 of the monitoring device can temporally retain drip chambers of such different shapes and/or different sizes, including drip chamber with different diameter and/or length.

FIG. 4a shows a top view of the fluid assist arrangement shown in FIG. 1. The retaining elements 14 are shaped with an inclination on the part of the retaining element being closest to the front side 5 of the body. The inclination shown starts from the body recess wall towards the part of the retaining element 14 protruding furthest out from the body recess wall. The inclination of the retraining elements 14 may allow a more effective arrangement of a drip chamber, as the force provided by inclined retraining elements 14 to the drip chamber increases as the drip chamber engages with the retaining elements 14.

FIG. 4b shows a top view of a variation of the fluid assist arrangement shown in FIG. 1. The retaining elements 14 are here shaped with a stem 14c connected to and protruding from the body recess wall 15 and a disc 14d adapted to be pressed against the drip chamber to temporally hold the drip chamber in fixed position in the body recess. The stem 14c may conveniently comprise a compressible coil spring that may be compressed when mounting the drip chamber in the body recess and may thereby press the opposite discs 14d against the drip chamber to hold the drip chamber stable and safe in the body recess.

FIG. 5 shows an arrangement comprising a monitoring device as shown in e.g. FIG. 1 and a fluid flow device 20. The monitoring device has a body recess 7 with a body recess axis BRa as illustrated. The body recess axis is illustrated as a dashed line and is an imaginary axis not visible to the human eye. Preferably, as shown in FIG. 5, the body recess axis BRa is arranged as a centered axis.

The fluid flow device 20 comprises a tube recess 21. The fluid flow device 20 is in the shown embodiment arranged below the monitoring device 3 and is connected to the body of the monitoring device 3. The tube recess 21 is adapted to fit a fluid tube. The fluid tube is not shown in FIG. 5. The fluid device also comprises a lid part 26 in an open configuration.

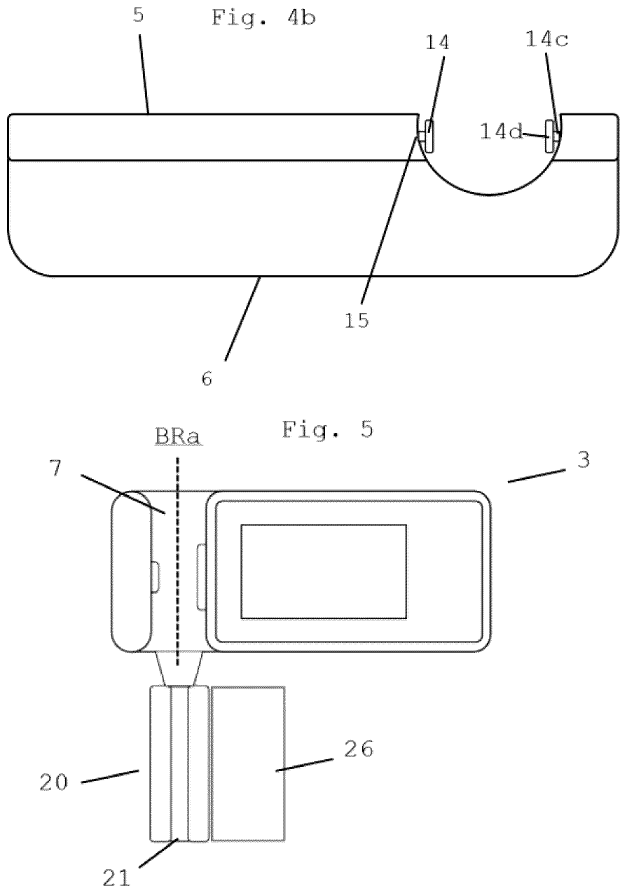

FIG. 6A shows a fluid flow device 20 with a fluid tube 2b arranged in the tube recess 21 (not visible). The tube recess is defined by a tube recess wall 21a in which the tube recess 21 is formed. Preferably a length section of the fluid tube 2b is located to be touching the surface 21b of the tube recess wall 21a. The tube recess wall further comprises sub-recess, preferably comprising a passage into the tube recess.

The fluid device comprises two air detectors 28, located in respective sub-recesses with a passage into the tube recess 21. The air detectors 28 are arranged at the tube recess 21 (not visible) to detect air or air bubbles in the fluid tube 2b. The fluid flow device 20 shown in FIG. 6A can be used in the shown configuration as well as upside down.

The fluid flow device 20 shown in FIG. 6A also comprises two pairs of force acting elements 19 at least partly located in respective sub-recess with a passage into the tube recess, such that the force acting elements 19 can apply a force to the fluid tube 2b. The force acting elements 19 of the pair of force acting elements 19 are arranged opposite to each other at least partly located in the respective sub-recess on each side of the tube recess and the fluid tube 2b. One of the pairs of force acting elements 19 comprises a constriction member 22 and an extending member 23. The constriction member 22 is arranged at the end of the extending member 23 and has a shape that allows the constriction member to constrict the fluid tube 2. Preferably, the constriction member 22 is made from a flexible material that prevents the fluid tube from being damaged when the constriction member exerts a force on the fluid tube. The extending member may comprise several parts that allow the extending member to extend through the passage into the tube recess in at least one direction.

If, and when one or both of the air detectors 28 detect air or air bubbles in the fluid, then a signal is sent through the assist controller (not shown) to at least one of the pairs of force acting elements 19 to constrict the tube and thereby stop the fluid from flowing in the fluid tube 2b.

The constriction members 22 may in an embodiment be adapted for use to change the fluid flow to another flow rate and/or velocity by constricting the fluid tube until the selected flow rate and/or velocity is obtained.

FIG. 6B illustrates an alternative to FIG. 6A, where the fluid flow device 20 comprises a force acting element 19 arranged partially in a sub-recess S and extending through the passage into the tube recess 21 in a tube distance Td from a protrusion member 27. In a loaded configuration, a fluid tube is arranged in the tube recess 21. The tube recess 21 may have a width which is smaller or wider compared to the tube distance Td. The protrusion member 27 allows the force acting element 19 to constrict the fluid tube by exerting a force on the fluid tube which is arranged against the protrusion member 27.

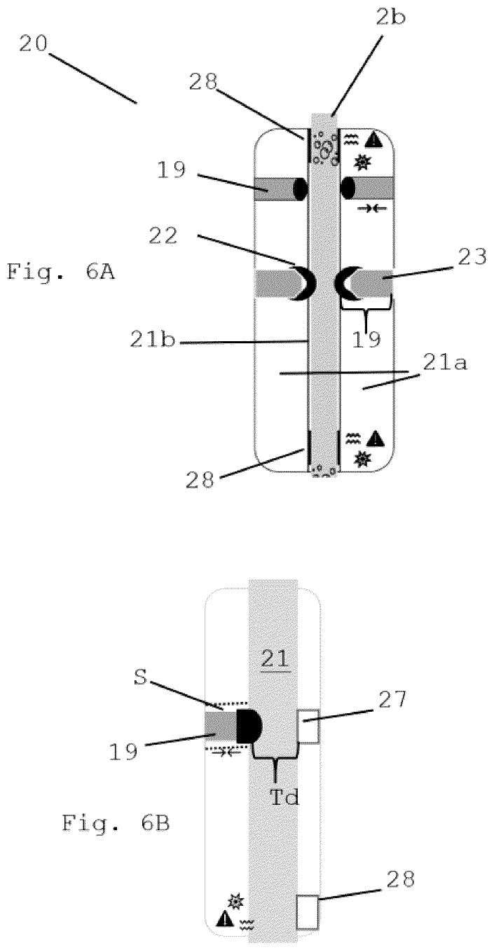

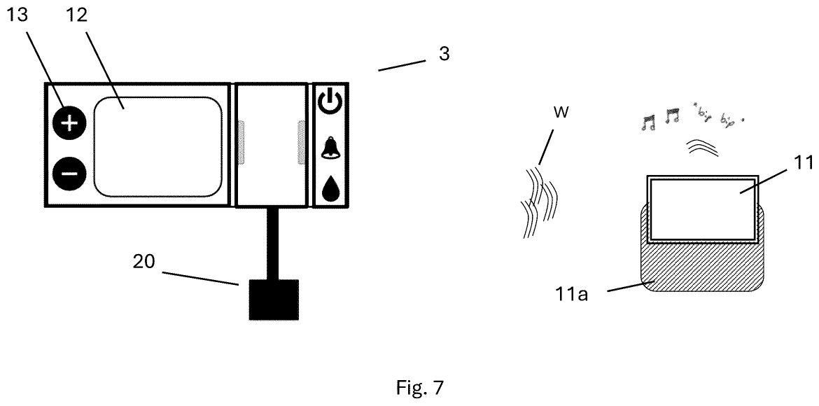

FIG. 7 is an alternative to FIG. 5, where the fluid assist arrangement comprises an assist controller 11, a monitoring device 3, comprising touch-sensitive buttons 13 and a fluid flow device 20. The assist controller 11 is in data connection, here shown as waves w, with the monitoring device 3 and the fluid flow device 20. The assist controller comprises an input arrangement 11a, here shown as a keyboard, which allows a user to provide input data to the assist controller 11.

FIG. 8A shows a fluid flow device comprising two force acting elements 19 arranged opposite to each other with the tube recess 21 in between. The force acting elements 19 comprises each an extending member 23 and a constriction member 22 rotatably connected to the extending member. The force acting elements 19 may be in a non-active mode where the force acting elements 19 are fully located in a sub-recess S of the tube recess wall as shown in FIGS. 8A and 8B, and the force acting elements 19 may be in an-active mode where at least a portion of the constriction member 22 is located in the tube recess for acting force onto a the fluid tube 2b when located in the tube recess.

FIG. 8B is an alternative to FIG. 8A, where fluid flow device comprises six force acting elements 19 arranged in pairs. The 3 pairs of force acting elements 19 allow the fluid flow device to constrict the fluid tube pair by pair and thereby an increased flow rate and/or velocity may be provided.

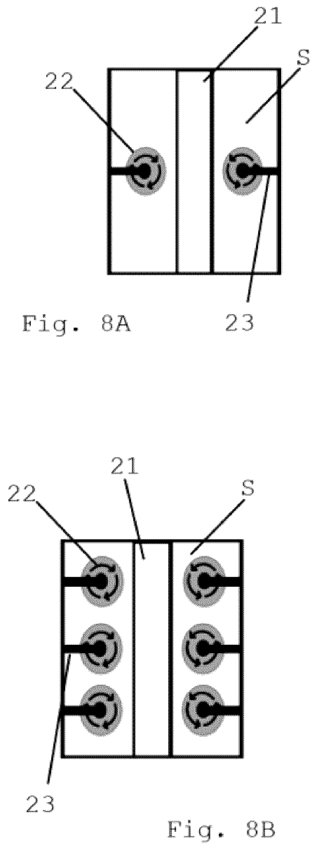

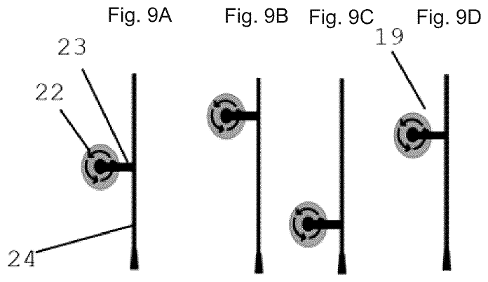

FIG. 9A-9D shows a force acting element connected to a threaded element 24 at different positions. The threaded element 24 is located in a sub recess S of the tube recess wall and is extending along a portion of the fluid flow device (not shown) with a passage into the tube recess. The force acting element 19 comprises a constriction member 22 rotatably connected to an extending member 23. The extending member 23 is also connected to the threaded element 24, so it can move in a longitudinal direction when the threaded element 24 rotates. FIG. 9A-9D shows different positions of the force acting element 19, when the threaded element has rotated either clockwise or anticlockwise. By providing a portion of the force acting elements 19 to extend through the passage into the tube recess, such that at least a portion of the constriction member 22 is are located in the tube recess to press towards the tube while simultaneously rotating the threaded element 24, to provide the force acting element 19 to move in a longitudinal direction while the constriction member 22 exert a pressure towards the fluid tube and rolls over the fluid tube, the force acting elements 19 may adjust the fluid flow in the fluid tube to a desired velocity. Thereby the force acting elements 19 may act both to constrict the fluid tube to reduce or fully stop a fluid flow through the fluid tube or the force acting elements 19 may increase the fluid flow through the fluid tube when the fluid tube 2b is located in the tube recess.

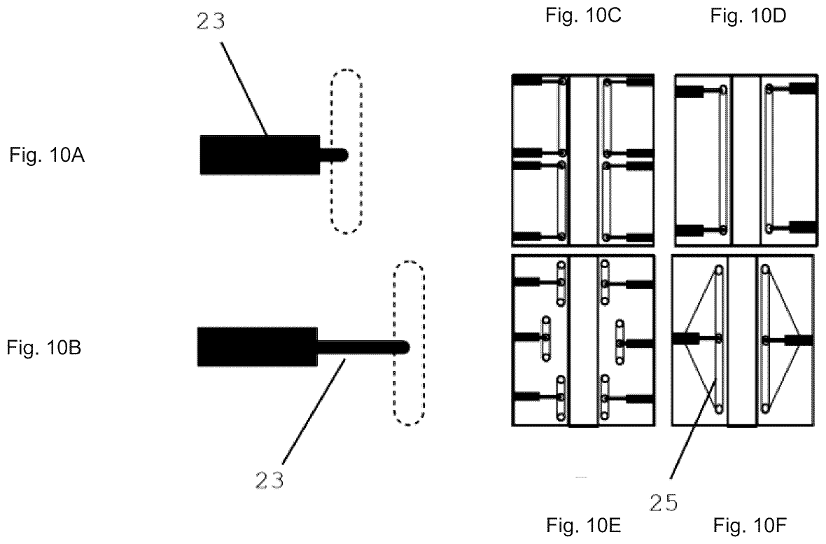

FIGS. 10A and 10B show an extending member 23 in a retracted position and an extended position. The extended member 23 may be connected to a constriction member with any shape as visually shown as a dashed object.

FIGS. 10C-10F show variations of FIGS. 8A and 8B. In FIG. 10F each of the force acting elements 19 comprises an extending member and constriction member connected to the extending member 23, where the constriction member comprises two elongate parts 25. The elongate parts 25 may allow the constriction member to have a shape which provides a selected constriction of the fluid tube and/or change of the flow rate and/or velocity of the fluid in the fluid tube.

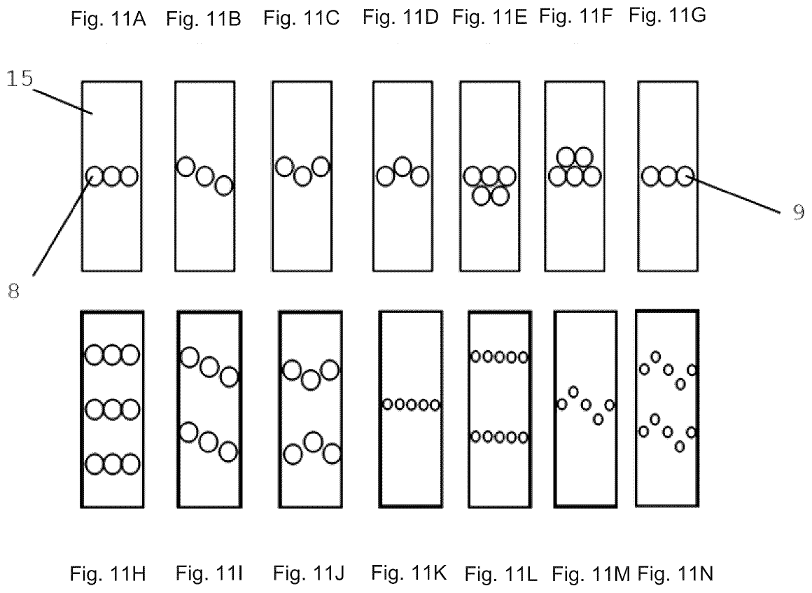

FIGS. 11A-11N show a tube recess wall 15 with different arrangements of one or more light emitting elements 8 and/or one or more light receiving elements 9. The light emitting elements 8 and/or the light receiving elements 9 may be arranged in pairs or in line to provide a coverage of a plane in the tube recess.

It has been found to be very beneficial where the one or more light emitting elements 8 comprises at least three light emitting elements 8 and wherein the one or more light receiving elements 9 comprises at least three light receiving elements, e.g. located as shown in FIGS. 11A and 11G. Thereby any tilting the fluid assist arrangement may immediately be registered and appropriate action may be taken. As described above, a such tilting may indicate that the patient is uncomfortable and/or dizzy, in which case a medical person, such as a nurse may immediately be alerted.

FIG. 12A shows a section of a body recess 7 with one light emitting element 8 and a light receiving element 9 arranged on the opposite side of the body recess 7 in different modes. In the first mode (1), the light emitting element 9 is turned off. In the second mode (2) the light emitting element 8 emits two light beams 10 across the drip chamber zone of the body recess 7. In the third mode (3) the light emitting element 8 emits one light beam across the drip chamber zone of the body recess 7. In the third mode, a beam axis Ba is shown. The beam axis is an imaginary central axis of the beam that indicates the direction of the beam. In the fourth mode (4) the light emitting element 8 emits a beam with a low beam divergence compared to the second mode (2) and the third mode (3).

FIG. 12B shows variations of FIG. 12A where both light emitting elements 8 and light receiving elements 9 are arranged on each side of the tube recess in different arrangements.

FIG. 13A shows a front view of an arrangement comprising a monitoring device 3.

The monitoring device 3 comprises a body recess 7 and a retaining arrangement comprising one retaining element 14 with two resilient arms 30. The resilient arms 30 extend along the length of the body recess 7, and each resilient arm 30 comprise two openings 17 arranged in a distance to each other. The resilient arms 30 embrace at least partially the drip chamber when the drip chamber is arranged in the body recess and thereby retain the drip chamber in a fixed position. The openings 17 are arranged to allow the light emitting elements to emit a light beam going through the drip chamber. The light emitting elements are not shown in FIG. 13A.

FIG. 13B shows from a top view an alternative of FIG. 1 and FIG. 13A, where the monitoring device comprises a retaining arrangement comprising two retaining elements arranged opposite to each other. Each of the retaining elements 14 comprises a curved shaped resilient arm 30. The shape of the resilient arms 30 allow the drip chamber to be retained in the body recess. The resilient arms 30 are made of a resilient material, which provides flexibility and allows the retaining arrangement to be used with different shaped drip chambers as for example shown in FIG. 18.

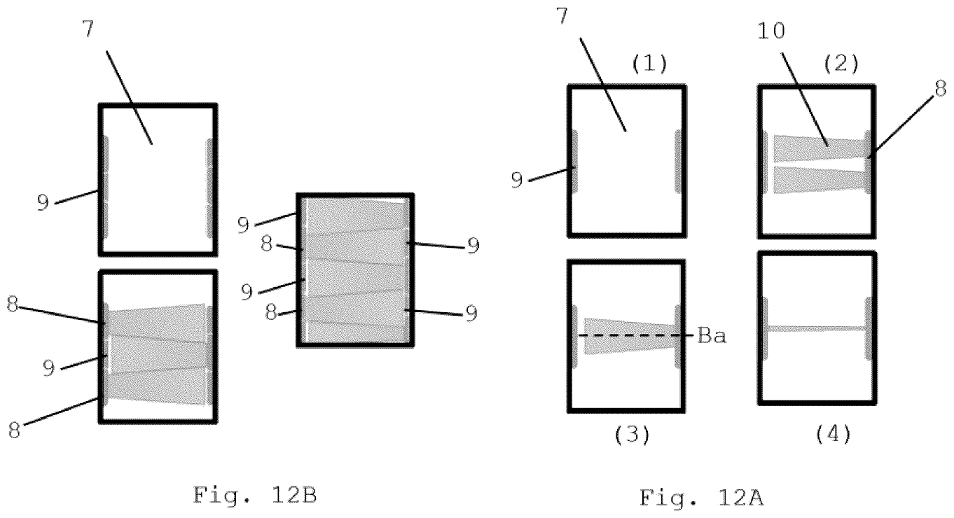

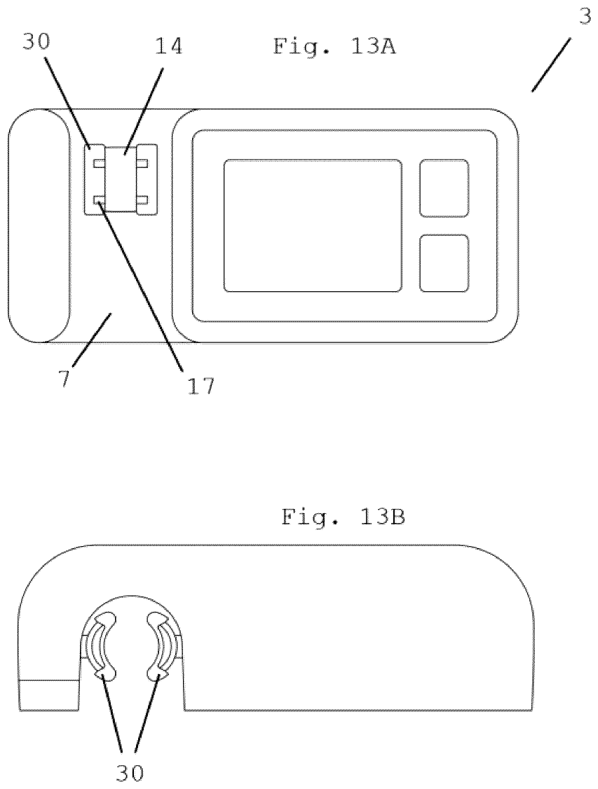

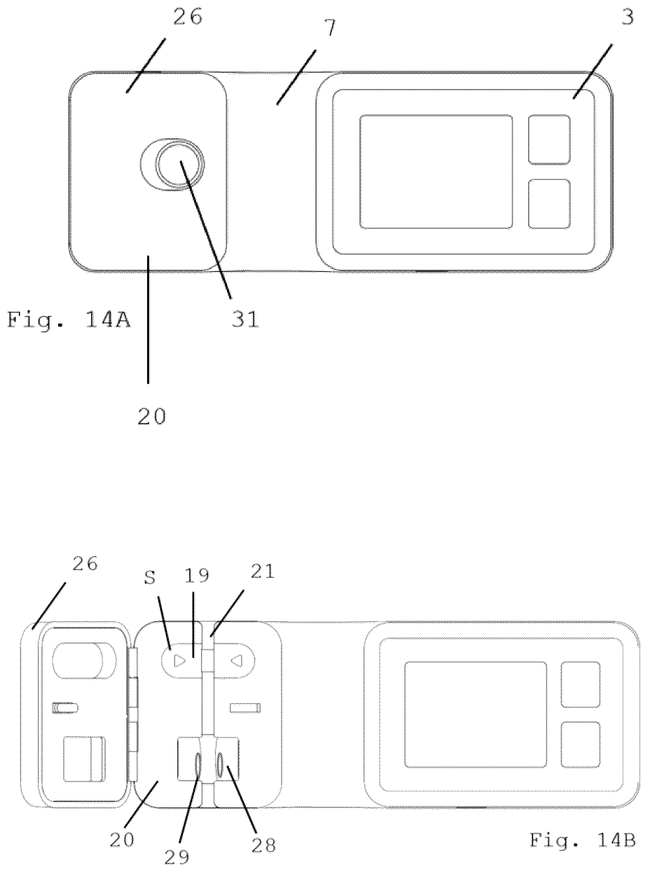

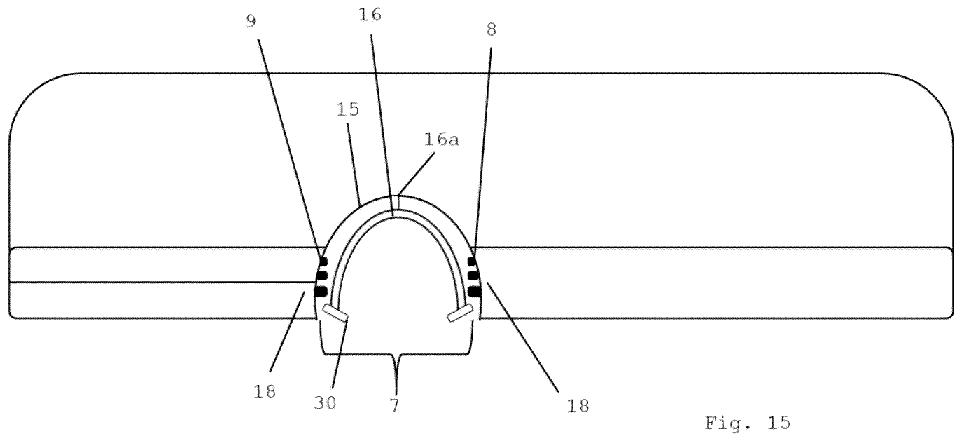

FIG. 14A shows a monitoring device 3 connected to a fluid flow device 20. The fluid flow device 20 comprises a lid part 26 in a closed configuration. The lid part is closed using a lock member 31, which allows the lid part 26 to remain in a closed configuration. The lock member 31 can be any locking mechanism, such as magnetic, spring operated, or snap lock mechanism.

FIG. 14B shows the fluid assist arrangement of FIG. 14A, where the top part 26 is in an open configuration. The fluid flow device 20 comprises an air detector 28 located in a sub-recess of the tube recess wall, which has an acoustic sensing element 29 arranged at the tube recess 21 in a sub-recess of the tube recess wall. The acoustic sensing element emits sound acoustic waves, which allow the acoustic sensing element 29 to detect air and/or air bubbles in a fluid.

The fluid flow device 20 also comprises force acting elements 19 arranged in a vertical distance from the acoustic sensing element 29 at the tube recess 21. The force acting elements 19 are located in sub-recesses S of the tube recess wall and are configured for constricting the fluid tube, if the acoustic sensing element 29 detects air and/or air bubbles in the fluid in the fluid tube, to prevent air and/or air bubbles from being dispensed with the fluid.

In the open configuration, air detector 28 and the force acting elements 19 are in an unloaded configuration, and the tube recess 21 may be loaded with a fluid tube. When the lid part 26 is locked into a closed configuration with a fluid tube accommodated in the tube recess, the fluid flow device is in a loaded configuration, where the force acting elements 19 may at least partially constrict the fluid tube to e.g. to retain the fluid flow device at the same position along a length of the fluid tube, to prevent the fluid flow device from sliding along the length of the fluid tube. As described above the force acting elements 19 may in addition be configured for adjusting the fluid flow in the fluid tube 2b.

FIG. 15 shows is a top view of a fluid assist arrangement comprising a monitoring device 3 with a body recess 7 having a curved tube recess wall 15. The monitoring device also comprises a retaining arrangement comprising one force generating element 16, which has two resilient arms 30 extending towards each other. The force generating element 16 is attached to the body recess wall 15 at a single location 15a and has two (not shown) openings for passage of one or more light beams emitted from the light emitting elements 8.

The monitoring device 3 of FIG. 15 comprises three light receiving elements 9 arranged at a first body recess section 18 side and three light emitting elements 8 arranged at a second body recess section 18 being opposite to the first body recess section 18. The light emitting elements 8 and the light receiving elements 9 are arranged together with the openings in a horizontal plane that is transverse to the longitudinal direction of the body recess.

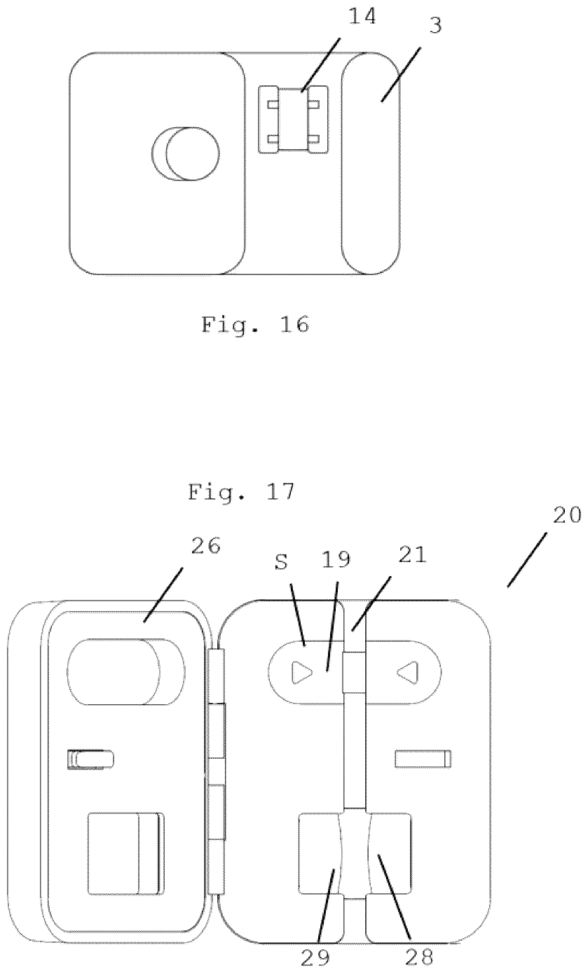

FIG. 16 shows an alternative monitoring device relative to the monitoring device of FIGS. 14A and 14B. In FIG. 16 the device 3 is illustrated with a retaining arrangement having a retaining element 14, and without a display unit 12.

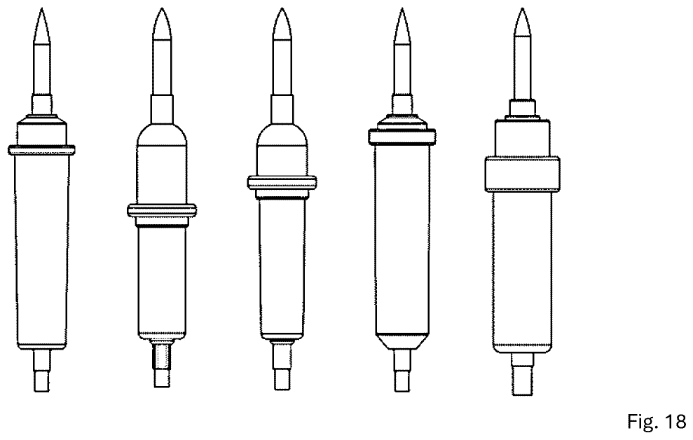

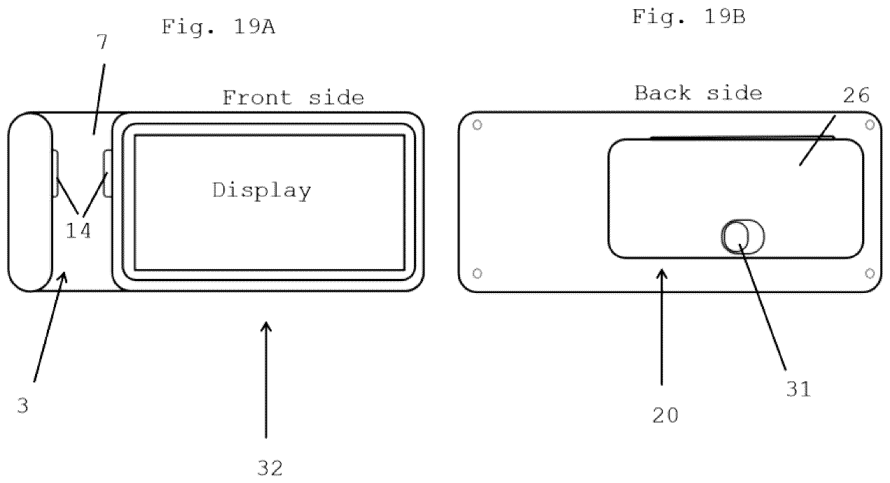

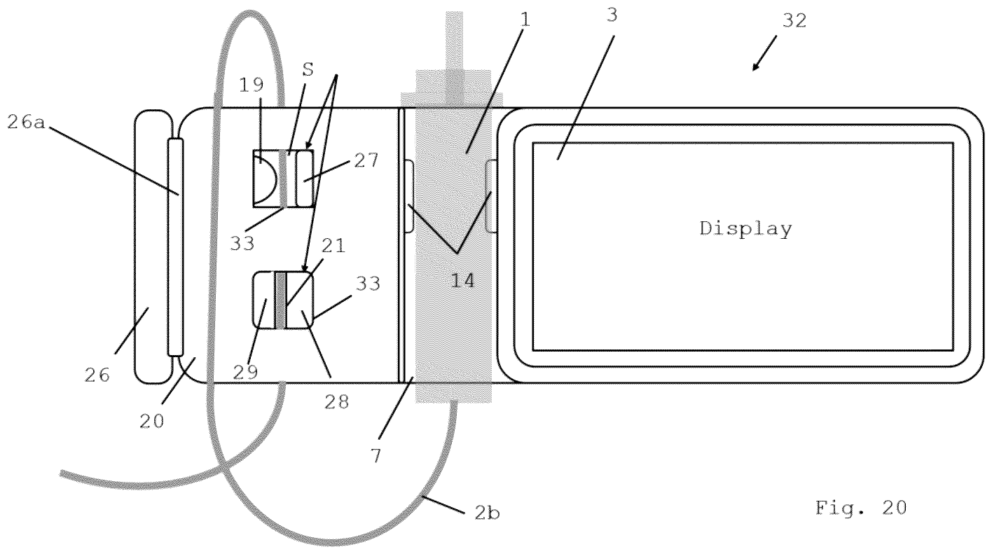

FIG. 17 shows a fluid assist arrangement device arrangement comprising a fluid flow device as shown in FIGS. 14A and 14B, but without a monitoring device.

FIG. 18 illustrates examples of five different drip chambers 1 that may be used with the arrangement of the invention. The shown drip chambers are typical drip chambers that are commercially available. A benefit of the fluid assist arrangement of the invention is that the fluid assist arrangement may be constructed to be capable of operate with any of these drip chambers. In addition the fluid assist arrangement of the invention may be constructed to operate with drip chambers of any drip factor (The number of drops (gtts) in one milliliter (mL) of fluid).

FIGS. 19A and 19B show a fluid assist arrangement of an embodiment of the invention seen from respectively a front side and a back side. The fluid assist arrangement 32 comprises a monitoring device 3 connected to a fluid flow device 20, wherein the monitoring device 3 is located at a front side of the fluid assist arrangement 32, meaning that it is accessible from the front side of the fluid assist arrangement 32. In FIG. 19A showing the front side we can see the body recess 7 and the retaining elements 14 The fluid flow device 20 is located at a back side of the fluid assist arrangement 32, meaning that it is accessible from the back side of the fluid assist arrangement 32. In FIG. 19B showing the back side of the fluid assist arrangement 32, we can see the lid part 26 comprising a lock member 31.

FIG. 20 shows a fluid assist arrangement 32 of an embodiment of the invention seem from the front side. The fluid assist arrangement 32 comprises a monitoring device 3 connected to a fluid flow device 20, wherein the monitoring device 3 is located at a front side of the fluid assist arrangement 32. The monitoring device comprises a body recess 7 comprising a pair of retaining elements 14. A drip chamber is located in the body recess 7 and temporally retained in the body recess by the pair of retaining elements 14. The body recess 7 and the retaining elements 14. The fluid flow device 20 is located at a back side of the fluid assist arrangement 32. The fluid assist arrangement comprises a pair of transparent windows 33 at the front side of the fluid assist arrangement 32 into the fluid flow device. The fluid flow device also comprises a lid part 26 and a hinge 26a for pivotally opening and closing the lid part 26 19B showing the back side of the fluid assist arrangement 32, we can see the lid part 26 comprising a lock member. A fluid tube 2b is connected to a bottom part of the drip chamber to collect fluid that has passes through the drip camber and for transporting it further to be infused to a patient from the drip chamber 1, the fluid tube 2 is passed upwards and to the backside of the fluid assist arrangement 32 from where it is inserted in to the tube recess 21, so that it is passing downwards through the fluid flow device 20. Through the upper most window the fluid tube can be seen at a location where sub-recesses S are passing into the tube recess 21 wherein these tube-recesses S comprises respectively force acting element 19 and protrusion member 7, wherein force acting element 19 is adapted to constrict the fluid tube 2b by exerting a force on the fluid tube which is arranged against the protrusion member 27.

As described above the potential risk of passing an air bubble to be infused to a patient may be practically avoided by this configuration.

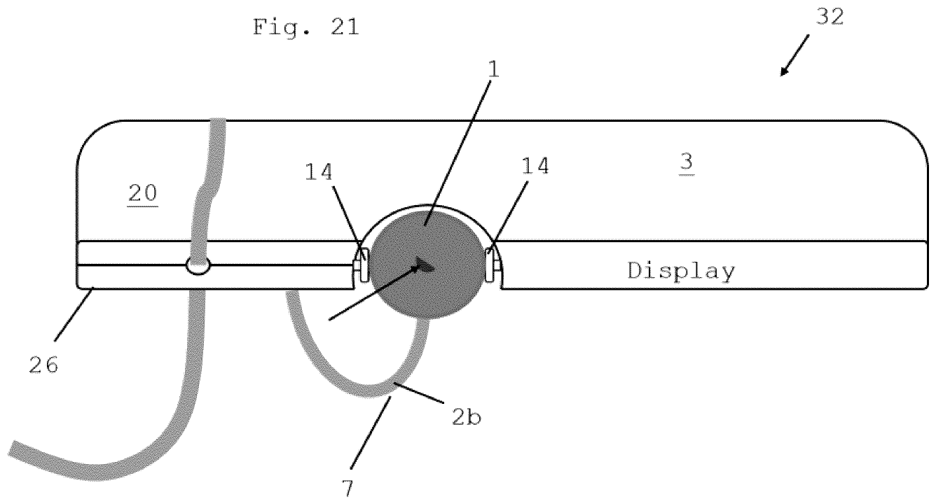

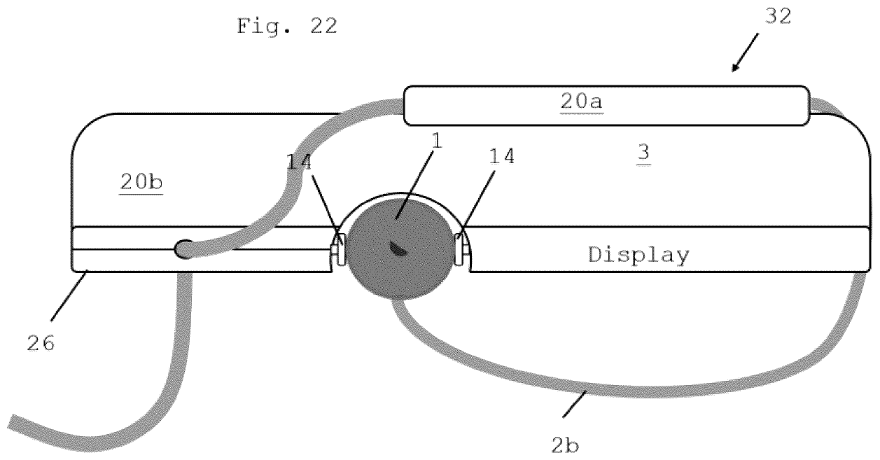

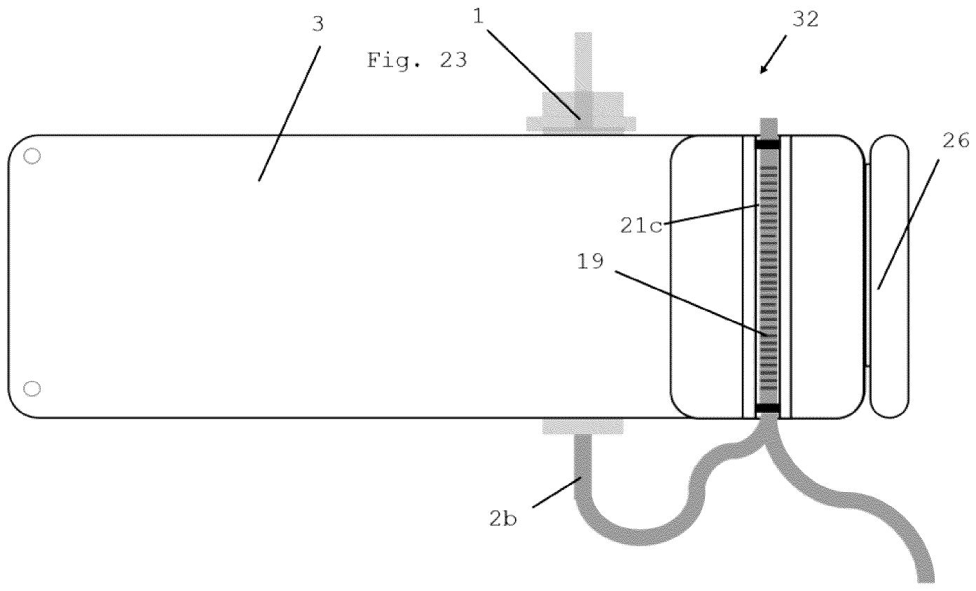

FIG. 21 shows a fluid assist arrangement 32 of another embodiment of the invention seen in a top view. The fluid assist arrangement 32 of FIG. 21, differs from the fluid assist arrangement 32 of FIG. 21 in that here the fluid flow device 20 is located at the front side of the fluid assist arrangement 32 side-by-side of the monitoring device 20.

In this embodiment the fluid tube 2b is connected to a bottom part of the drip chamber to collect fluid that has passes through the drip camber and for transporting it further upwards along the backside of the fluid assist arrangement 32 to the top of the fluid assist arrangement and downwards through the tube recess to be infused to a patient.

As described above the potential risk of passing an air bubble to be infused to a patient may be practically avoided by this configuration.

FIG. 22 shows a variation of the fluid assist arrangement 32 FIG. 21, wherein the fluid assist arrangement comprises two fluid flow devices 20a, 20b.

A first fluid flow device 20a of the fluid flow devices. In the tube recess is located horizontally. This fluid flow device may conveniently comprise a not shown air detector and/or a not shown crystallized matter sensor. Thereby the fluid flow may be stopped very fast, should an air bubble or crystallized matter ne detected. The second fluid flow device 20b is vertically orientated and may conveniently comprise one or more force acting elements, such as the force acting elements shown in FIGS. 8A, 8B and/or FIG. 9.

In this embodiment the fluid tube 2b is connected to a bottom part of the drip chamber to collect fluid that has passes through the drip camber and for transporting it further onto the and horizontally along the backside where it is passing through the first fluid flow device 20a and further upwards to the top of the fluid assist arrangement and downwards through the tube recess of the second fluid flow device 10b to be infused to a patient.

FIG. 23 shows a variation of the fluid assist arrangement 32 FIG. 20. The fluid assist arrangement 32 is seen from the back side and comprises a monitoring device 3 with a drip chamber 1 at the front side. The fluid assist arrangement comprises a fluid flow device 20 with a double tube recess. 21c

The fluid tube 2b is connected to a bottom part of the drip chamber 1 to collect fluid that has passes through the drip camber and for transporting it further to the backside of the fluid assist arrangement 32 and upwards through a first section of the fluid tube 2b to the top of the fluid assist arrangement and downwards through a second section of the fluid tube 2b and further to be infused to a patient. Both of the first section of the fluid tube 2b and the second section of the fluid tube 2b are located in the double tube recess 21c of the fluid flow device 20. The first section of the fluid tube 2b and the second section of the fluid tube 2b may be located in the double tube recess 21c is in a side by side configuration, e.g. such that an air detector is located to detect air in the first section of the fluid tube 2b and a plurality og force generating elements 19 are located to act on the second section of the fluid tube 2b.

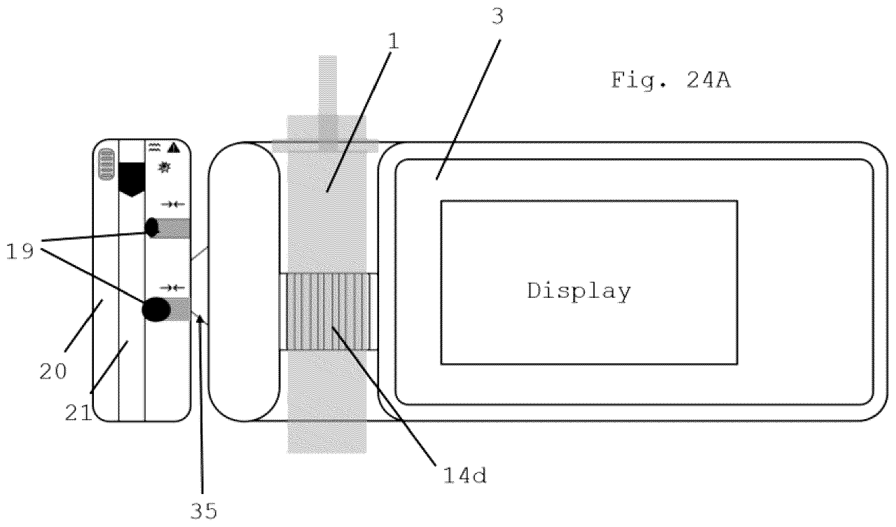

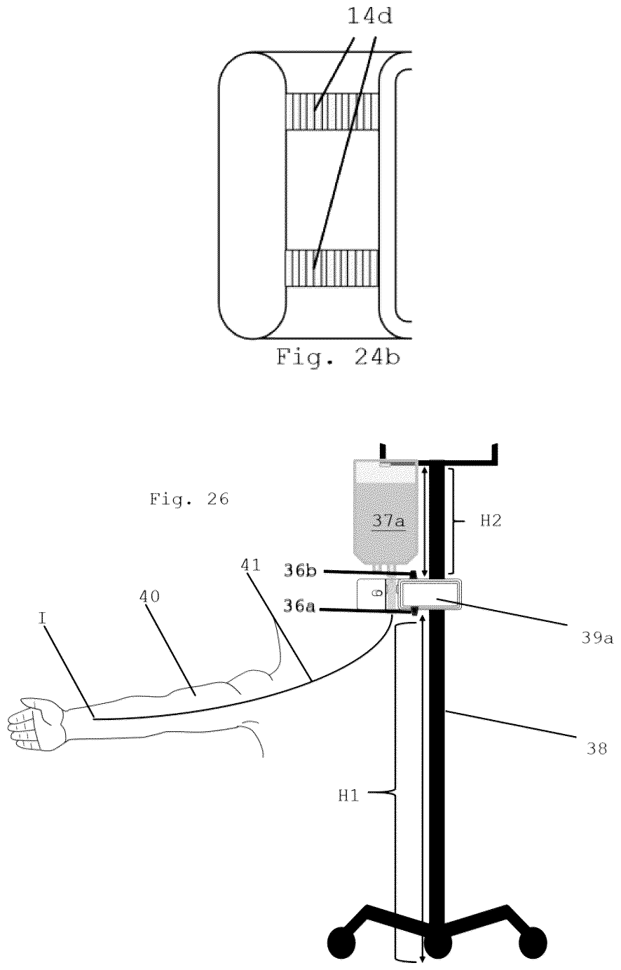

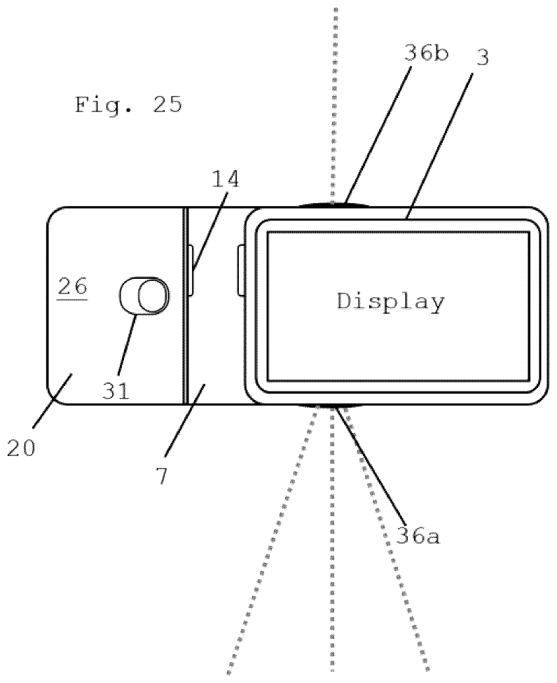

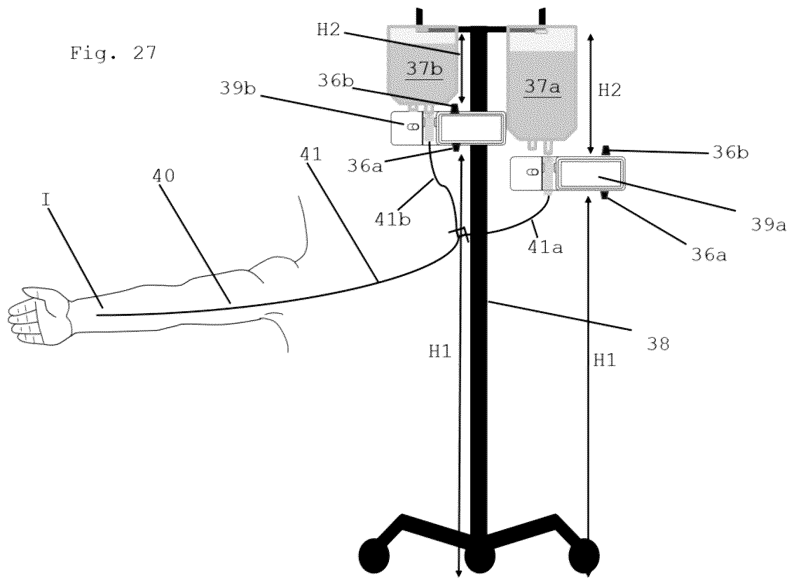

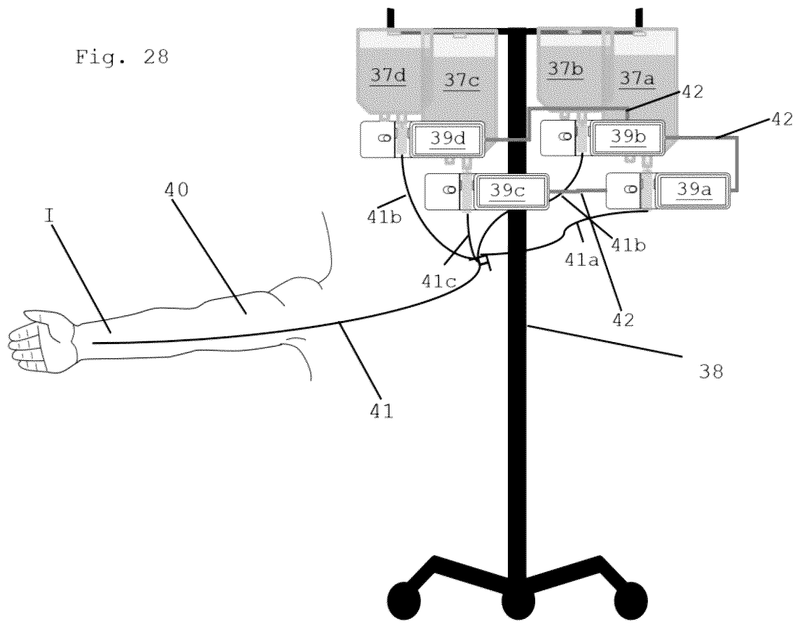

FIG. 24A illustrates a fluid assist arrangement of an embodiment of the invention where the retaining element is in the form of an elastic band 14d. The fluid assist arrangement comprises monitoring device 3 with a body recess 7 and a drip chamber 1 temporally retained in the body recess 7 by the retaining element which here is in the form of an elastic band 14d. In the shown embodiment a fluid flow device 20 is mounted to the monitoring device 3 via a mounting arrangement 35, which preferably may be a click-om mounting arrangement, preferably the mounting arrangement 35 comprises a mechanical fastening or attachment system that allows the fluid flow device 20 to be easily secured to and detached from the monitoring device 3.

FIG. 24B illustrates a portion of the fluid assist arrangement of FIG. 14A, where the single elastic band 14d has been replaced by a pair of elastic bands 14d.

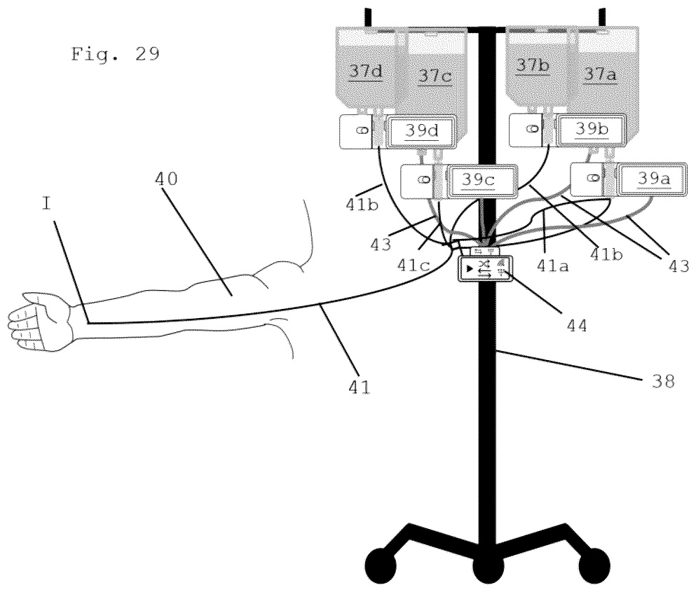

FIG. 25 illustrates a fluid assist arrangement of an embodiment of the invention comprising at least one TOF sensor. The fluid assist arrangement comprises a monitoring device 3 connected to a fluid flow device 20. The fluid flow device 20 comprises a lid part 26 in a closed configuration. The lid part is closed using a lock member 31, which allows the lid part 26 to remain in a closed configuration for a desired period. The monitoring device 3 comprises a body recess 7 comprising retaining elements for retaining a drip chamber.

The fluid assist arrangement comprises a first TOF sensor 36a located to pointing downwards from the monitoring device 3. In the shown embodiment the first TOF sensor 36a is located to pointing downwards from the fluid assist arrangement and the first TOF sensor is mounted to and/or fixed to a lower edge of the monitoring device 3.

The fluid assist arrangement comprises a second TOF sensor 36b located to pointing upwards from the monitoring device 3. In the shown embodiment the first TOF sensor 36a is located to pointing upwards from the fluid assist arrangement and the first TOF sensor is mounted to and/or fixed to an upper edge of the monitoring device 3.

The first TOF sensor 36a and the second TOF sensor 36b may independently of each other be as described above.

FIG. 26 illustrates a fluid assist installation comprising a fluid assist arrangement 39a of an embodiment of the invention. The installation comprises an IV pole 38 and a fluid dispensing module comprising a fluid assist arrangement 39a according to an embodiment of the invention, an IV bag 37a with an infusion fluid and an IV fluid system comprising a drip chamber and a fluid tube in fluid connection with the drip chamber. The infusion fluid of the IV bag 37a is to be dispensed to a patient. The IV bag 37a is connected to the drip chamber of the IV fluid system and is retained in the fluid assist arrangement 39a. The fluid assist arrangement 39a comprises a first TOF sensor 36a and a second TOF sensor 36b. A fluid tube 41 is located to transport the infusion fluid from the drip chamber to be infused to an arm 40 of a patient. The first TOF sensor 36a is located to determine the height distance H1 from the fluid assist arrangement 39a to the floor and the second TOF sensor 36b is located to determine the height distance H2 from the fluid assist arrangement 39a to a location of the IV pole 38, in this embodiment a hook of the IV pole 38 onto which the IV 37a is hanging. The first TOF sensor 36a and the second TOF sensor 36b may therefore be applied for determining an exact height position of the drip chamber and the inlet to the fluid tube 41 e.g. relative to an infusion site I of injecting the infusion fluid to the patients arm 40

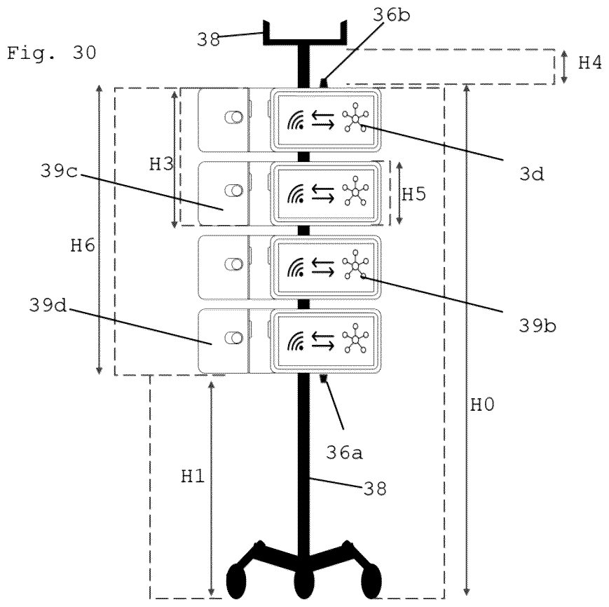

FIG. 27 illustrates a fluid assist installation of an embodiment of the invention. The installation comprises a plurality of fluid dispensing modules comprising a first fluid dispensing module comprising a first fluid assist arrangement 39a of an embodiment of the invention and a second fluid dispensing module comprising second fluid assist arrangement 39b of an embodiment of the invention, wherein the first fluid assist arrangement 39a and the second fluid assist arrangement 39b may be equal or different from each other.

The first fluid dispensing module comprises the first fluid assist arrangement 39a, a first IV bag 37a with an infusion fluid and a first IV fluid system comprising a drip chamber and a fluid tube in fluid connection with the drip chamber to transport the infusion fluid of the first IV bag 37a to the drip chamber. The first IV bag 37a is connected to the drip chamber of the first IV fluid system and is retained in the body recess of the first fluid assist arrangement 39a.

The second fluid dispensing module comprises the second fluid assist arrangement 39b, a second IV bag 37b with an infusion fluid and a second IV fluid system comprising a drip chamber and a fluid tube in fluid connection with the drip chamber to transport the infusion fluid of the second IV bag 37b to the drip chamber. The second IV bag 37b is connected to the drip chamber of the second IV fluid system and is retained in the body recess of the second fluid assist arrangement 39b.

Each of the first fluid assist arrangement 39a the second fluid assist arrangement 39b comprises a first TOF sensor 36a and a second TOF sensor 36b to determine respectively the height distance H1 and the height distance H2 of respectively the first fluid assist arrangement 39a the second fluid assist arrangement 39b and are preferably configured for determining an exact height position of the drip chamber retained in the body recess of respectively the first fluid assist arrangement 39a the second fluid assist arrangement 39b.

The fluid assist installation of the embodiment of FIG. 27 comprises a delivery tube arrangement comprising a first pre-supply tube section 41a, a second pre-supply tube section 41b and a main supply tube section 41, wherein the first pre-supply tube 41a section is connected to receive the infusion fluid from the drip chamber of the first IV fluid system and deliver the infusion fluid to an inlet of the main supply tube section 41, the second pre-supply tube section 41b is connected to receive the infusion fluid from the drip chamber of the second IV fluid system and deliver the infusion fluid to the inlet of the main supply tube section 41 and wherein the main supply tube section 41 is located to transport the infusion fluid received from the first pre-supply tube section 41a and the second pre-supply tube section 41b and deliver the infusion fluid to be infused at an infusion site I to an arm 40 of a patient.

FIG. 28 illustrates another fluid assist installation of an embodiment of the invention. Here the fluid assist installation comprises four fluid dispensing modules. The first fluid dispensing module comprises the first fluid assist arrangement 39a, a first IV bag 37a with an infusion fluid and a first IV fluid system comprising a drip chamber and a fluid tube in fluid connection with the drip chamber to transport the infusion fluid of the first IV bag 37a to the drip chamber of the first IV fluid system. The second IV bag 37b is connected to the drip chamber of the second IV fluid system and is retained in the body recess of the second fluid assist arrangement 39b. The second fluid dispensing module comprises the second fluid assist arrangement 39b, a second IV bag 37b with an infusion fluid and a second IV fluid system comprising a drip chamber and a fluid tube in fluid connection with the drip chamber to transport the infusion fluid of the second IV bag 37b to the drip chamber of the second IV fluid system. The second IV bag 37b is connected to the drip chamber of the second IV fluid system and is retained in the body recess of the second fluid assist arrangement 39b. The third IV bag 37c is connected to the drip chamber of the third IV fluid system and is retained in the body recess of the third fluid assist arrangement 39c. The third fluid dispensing module comprises the third fluid assist arrangement 39c, a third IV bag 37c with an infusion fluid and a third IV fluid system comprising a drip chamber and a fluid tube in fluid connection with the drip chamber to transport the infusion fluid of the third IV bag 37c to the drip chamber of the third IV fluid system. The third IV bag 37c is connected to the drip chamber of the third IV fluid system and is retained in the body recess of the third fluid assist arrangement 39c. The fourth IV bag 37d is connected to the drip chamber of the fourth IV fluid system and is retained in the body recess of the fourth fluid assist arrangement 39d. The fourth fluid dispensing module comprises the fourth fluid assist arrangement 39d, a fourth IV bag 37d with an infusion fluid and a fourth IV fluid system comprising a drip chamber and a fluid tube in fluid connection with the drip chamber to transport the infusion fluid of the fourth IV bag 37d to the drip chamber of the fourth IV fluid system. The fourth IV bag 37d is connected to the drip chamber of the fourth IV fluid system and is retained in the body recess of the fourth fluid assist arrangement 39b

The fluid assist installation of the embodiment of FIG. 28 comprises a delivery tube arrangement comprising a first pre-supply tube section 41a, a second pre-supply tube section 41b, a third pre-supply tube section 41c, a fourth pre-supply tube section 41d and a main supply tube section 41, wherein the first pre-supply tube 41a section is connected to receive the infusion fluid from the drip chamber of the first IV fluid system and deliver the infusion fluid to an inlet of the main supply tube section 41, the second pre-supply tube section 41b is connected to receive the infusion fluid from the drip chamber of the second IV fluid system and deliver the infusion fluid to the inlet of the main supply tube section 41, the third pre-supply tube section 41c is connected to receive the infusion fluid from the drip chamber of the third IV fluid system and deliver the infusion fluid to the inlet of the main supply tube section 41, the fourth pre-supply tube section 41d is connected to receive the infusion fluid from the drip chamber of the fourth IV fluid system and deliver the infusion fluid to the inlet of the main supply tube section 41 and wherein the main supply tube section 41 is located to transport the infusion fluid received from the first pre-supply tube section 41a, the second pre-supply tube section 41b, third pre-supply tube section 41c and the fourth pre-supply tube section 41d and to deliver the infusion fluid to be infused at an infusion site I to an arm 40 of a patient.

The infusion fluid of respectively the first IV bag 37a, the second IV bag 37b, the third IV bag 37c and the fourth IV bag 37d may differ from each other and as described above it may be important that the volumes of infusion fluid supplied to the patient of the respective infusion fluids are adjusted relative to each other. Therefore, in order to adjust the volume of infusion fluid dispensed to the patient from the respective fluid dispensing modules, the first fluid assist arrangement 39a, the second fluid assist arrangement 39b, the third fluid assist arrangement 39c and the fourth fluid assist arrangement 39d are in wired data communication via the wires 42.

In a variation thereof the respective fluid dispensing modules are in wireless data communication e.g. as described above.

FIG. 29 illustrates yet another fluid assist installation of an embodiment of the invention. The embodiment of FIG. 29 differs from the embodiment of FIG. 28 in that the fluid assist installation comprises a central controller 44 and in that the first fluid assist arrangement 39a, the second fluid assist arrangement 39b, the third fluid assist arrangement 39c and the fourth fluid assist arrangement 39d are in wired data communication with the central controller 44. Thereby the fluid dispensing modules may be in data communication for adjusting the operation of the respective fluid dispensing modules.

In a variation thereof the respective fluid dispensing modules are in wireless data communication e.g. as described above.

The fluid assist installation shown in FIG. 30 comprises a rack of fluid assist arrangements, here comprising four fluid assist arrangements 39a, 39b, 39c, 39d and an IV pole 38, wherein the four fluid assist arrangements 39a, 39b, 39c, 39d are temporally or stationary fixed to the IV pole 38 below the one or more IV hooks 38a of the IV pole 38. The four fluid assist arrangements 39a, 39b, 39c, 39d are temporally or stationary fixed to the IV pole 38 with a known height distance to each other. The lowermost fluid assist arrangement 39a of the fluid assist arrangements 39a, 39b, 39c, 39d comprises a first TOF sensor 36a arranged to detect the height distance H1 to the floor and the uppermost fluid assist arrangement 39d of the fluid assist arrangements 39a, 39b, 39c, 39d comprises a second TOF sensor 36b 36a arranged to detect the height distance H4 to the one or more IV hooks.

Since the four fluid assist arrangements 39a, 39b, 39c, 39d have identical heights all relevant heights of the fluid assist installation may now be determined, including the height distance H0 from the floor to the upper edge of the uppermost fluid assist arrangement 39d, the height distance H1 from the floor to the lower edge of the lowermost fluid assist arrangement 39d, the height H5 of each of the respective fluid assist arrangements 39a, 39b, 39c, 39d and the height distances between any of the fluid assist arrangements 39a, 39b, 39c, 39d, such as the height distance H3 between the upper edge of the uppermost fluid assist arrangement 39d and a lower edge of the fluid assist arrangements 39c immediately below of the uppermost fluid assist arrangement 39d.

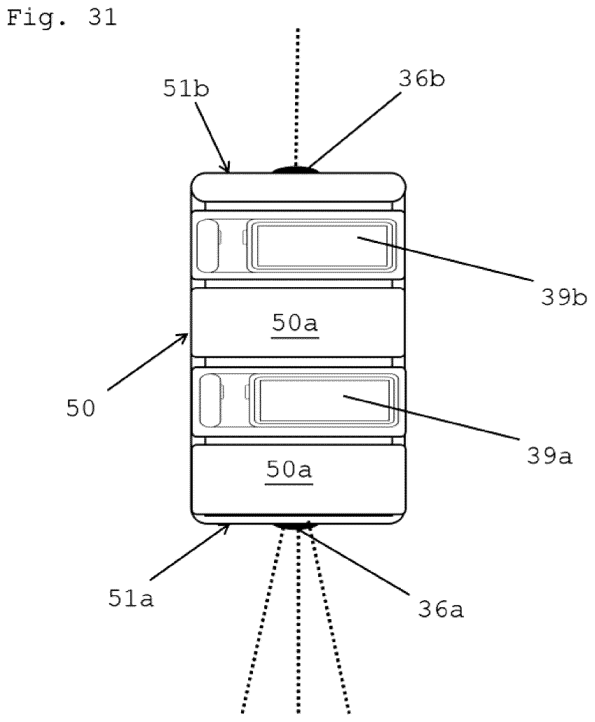

The fluid assist installation shown in FIG. 31 comprises a rack comprising one or more fluid assist arrangements. In the shown embodiment the rack 50 comprises four shelfs 50a, wherein in number 2 and 4 from a bottom surface 51a to the top surface 51b a first fluid assist arrangement 39a and a second fluid assist arrangement 39b are located. In total. the shown rack 50 has space for four fluid assist arrangements. In variations thereof, the rack may have space for two, three, five or more fluid assist arrangements. Thereby the rack may be used in a very flexible manner and t is simple to replace a fluid assist arrangement should it be required.

The rack 50 may e.g. be mounted to an IV pole below one or more IV hooks of the IV pole.

The rack 50 comprises a first TOF sensor 36a located at its a bottom surface 51a to detect a height distance to the floor and a second TOF sensor 36b located at its a top surface 51b to detect a height distance e.g. to a ceiling and/one or more IV hooks of an IV pole

The first TOF sensor 36a and the second TOF sensor 36b ensures that any relevant heights of the respective fluid assist arrangements 39a, 39b located in the rack 50 may be determined with a high accuracy.

1. A fluid assist arrangement for determining at least one fluid characteristic of a fluid in a fluid system comprising a drip chamber and a fluid tube in fluid connection with the drip chamber, wherein the fluid assist arrangement comprises an assist controller and a monitoring device, wherein the monitoring device comprising
a body having a front side and a back side, wherein the front side comprises a body recess defining a body recess volume, and wherein the body recess is adapted to accommodate at least a portion of a drip chamber,
one or more light emitting elements arranged to emit at least one light beam to pass at least a portion of the body recess volume, wherein the at least one light beam has a beam axis,
one or more light receiving elements are configured to receive at least a portion of said at least one light beam and to convert said portion of said at least one light beam into an electrical signal,
wherein the assist controller is in data communication with at least the one or more light receiving elements and is adapted to receive the electrical signal and wherein the assist controller is configured for determining at least one fluid characteristic of the fluid by the received electrical signal.

2. The fluid assist arrangement according to claim 1, wherein the at least one fluid characteristic comprises at least one of a velocity, a flow rate, a temperature, a turbidity, a concentration, a pH value, and/or a volume flow.

3. The fluid assist arrangement according to claim 1, wherein the monitoring device comprises a retaining arrangement for temporally retaining a drip chamber in the body recess, wherein the retaining arrangement comprises at least one retaining element at least partially located in the body recess, wherein the at least one retaining element is at least partially made from a resilient polymercomprising a polymer having a modulus of resilience Uᵣ of at least about 0.3 MPa determined according Yield strength/2*Youngs modulus, determined according to ISO 527-1:2019.

4. The fluid assist arrangement according to claim 1, wherein the monitoring device comprises a retaining arrangement for temporally retaining a drip chamber in the body recess, wherein the retaining arrangement comprises at least one retaining element at least partially located in the body recess, wherein the one or more openings is arranged to allow the one or more light receiving elements to receive at least a portion of the at least one beam through the one or more openings.

5. The fluid assist arrangement of claim 1, wherein the one or more light emitting elements are located to emit the at least one light beam to pass through a drip chamber zone of the body recess volume and wherein at least one of the one or more light receiving elements are located in a light receiving chamber of the body recess wall and wherein the body has a light passage from the drip chamber zone to the at least one of the one or more light receiving located in the light receiver chamber to receive at least a portion of said at least one light beam that has passed through the drip chamber zone of the body recess volume.

6. The fluid assist arrangement according to claim 1, wherein the body recess comprises a body recess axis and wherein the at least one light beam illuminates more than 50% of the body recess volume in a plane perpendicular to the body recess axis.

7. The fluid assist arrangement according to claim 1, wherein the monitoring device comprises at least one optical filter located to filter the light beam between said one or more light emitting elements and said one or more light receiving elements, wherein said at least one optical filter comprises at least one of a band-pass filter, a long-pass filter, a short-pass filter, a dichroic filter and/or a polarized filter.

8. The fluid assist arrangement according to claim 1, wherein the fluid assist arrangement comprises an orientation sensor adapted for determining tilt of the fluid assist arrangement.

9. The fluid assist arrangement according to claim 1, wherein the fluid assist arrangement comprising at least one fluid flow device for adjusting a liquid flow in a fluid tube, wherein the fluid assist arrangement comprising one or more force acting elements, wherein the one or more force acting elements are configured for adjustably constricting the fluid tube to adjust the liquid flow in the fluid tube and wherein each of the one or more force acting elements comprise a constriction member connected to an extending member, wherein the constriction member is adapted to engage with the fluid tube.

10. The fluid assist arrangement according to claim 9, wherein the at least one fluid flow device comprises a tube recess, and wherein the one or more force acting elements are arranged at one or more sub-recesses in a tube recess wall having a passage into the tube recess and wherein at least a part of the extending member is located in the at least one sub-recess of the tube recess and wherein at least a portion of the constriction member is adapted to be located in the tube recess when in operation to constrict the fluid tube, wherein the force acting elements is configured for extend the extending member to pass at least the portion of the constriction member through said recess passage into said tube recess to constrict said fluid tube.

11. The fluid assist arrangement according to claim 9, wherein the constriction member is rotatably connected to the extending member, wherein the at least one fluid flow device comprises at least one threaded element located in the at least one sub-recess and extending along a length of the at least one fluid flow device, and wherein the extending member of each of the one or more force acting element is connected to the threaded element and adapted to move along the length of the threaded element when the threaded element is rotated.

12. The fluid assist arrangement according to claim 9, wherein the constriction member comprises at least one elongate part, wherein the at least one elongate part is adapted for performing tilting motions to constrict the fluid tube gradually along a length of the fluid tube to pump the fluid in a desired direction.

13. The fluid assist arrangement according to claim 9, wherein the one or more force acting elements of the at least one fluid flow device comprises at least a pair of force acting elements arranged opposite to each other, wherein each of the force acting elements of a pair of force acting elements are located opposite to each other and are moving mirrored to each other.

14. The fluid assist arrangement according to claim 9, wherein the fluid assist arrangement comprises an air detector, wherein the air detector comprises an acoustic sensing element adapted to detect air in the fluid tube and wherein the assist controller is configured for controlling said one or more force acting elements at least partially in dependence of the air detector.

15. The fluid assist arrangement according to claim 14, wherein the assist controller is configured for submitting an electrical signal to the at least one fluid flow device to provide at least one of the of the one or more force acting elements to constricting the fluid tube to terminate the fluid flow in the fluid tube at least temporally.

16. The fluid assist arrangement according to claim 1, wherein the fluid assist arrangement comprises at least one time-of flight (TOF) sensor.

17. The fluid assist arrangement according to claim 16, wherein the at least one TOF sensor comprises a first TOF sensor adapted for determining a height location of the fluid assist arrangement., wherein the first TOF sensor is located to pointing downwards from at least one of the monitoring device and/or from the fluid flow device when the fluid assist arrangement is in use and/or wherein the at least one TOF sensor comprises a second TOF sensor adapted for determining a distance to an upwards located element, wherein the second TOF sensor is located to pointing upwards from at least one of the monitoring device and/or the fluid flow device when the fluid assist arrangement is in use.

18. The fluid assist arrangement according to claim 16, wherein the fluid assist arrangement is configured for being in data communication with at least one other fluid assist arrangement and/or with a central controller, wherein said fluid assist arrangement is configured for being in data communication with the at least one other fluid assist arrangement and/or with the central controller by a wired connection and/or by a wireless connection.

19. The fluid assist arrangement according to claim 1, wherein the fluid assist arrangement comprises transmitter, a receiver and/or a transceiver adapted for being in data communication with at least one other fluid assist arrangement and/or with a central controller.

20. A fluid assist installation, wherein the fluid assist installation comprises a plurality of fluid dispensing modules, wherein each fluid dispensing module comprises a fluid assist arrangement according to claim 1, wherein the fluid assist arrangement of one or more of the plurality of fluid dispensing modules is in data communication with the fluid assist arrangement of at least one other of the plurality of fluid dispensing modules and/or is in data communication with a central controller.

21. The fluid assist installation of claim 20, wherein each of the plurality of fluid dispensing modules comprises an IV bag and a fluid system comprising a drip chamber and a fluid tube in fluid connection with the drip chamber, wherein the fluid assist arrangement is arranged for assisting dispensing of a fluid from the IV bag via the fluid system, wherein the drip chamber is at least partly located in the body recess of said fluid assist arrangement.

22. The fluid assist installation of claim 20, wherein the IV bag of each of the one or more of said plurality of fluid dispensing modules is hanging at an IV pole and/or wherein the IV bag of each of the one or more of said plurality of fluid dispensing modules is located in a dispensing pump.

23. A flow device arrangement for assisting dispensing of a fluid in a fluid system comprising a fluid tube, wherein the arrangement comprises an assist controller and a fluid flow device comprising
a main housing comprising a tube element adapted to accommodate a fluid tube, comprising a tube recess, and
one or more force acting elements arranged at the tube element,
wherein the one or more force acting elements are configured for adjustably constricting the fluid tube to adjust a fluid flow in the fluid tube.

24. The flow device arrangement according to claim 23, wherein the fluid assist arrangement comprises an air detector and/or a crystallized matter detector arranged at the tube element, wherein the air detector comprises an acoustic sensing element adapted to detect air in the fluid tube and the crystallized matter detector comprises an optical detector.
